SEQUENCE LISTING (10 sequences)

SEQ ID NO: 1 - protein, 1114 aa, Artificial Sequence
  fusion protein
QVQLQESGGGSVQAGGSLKLTCAASGYIFNSCGMGWYRQSPGRERELVSRISGDGDTWHKESVKGRFTISQDNVKKTLYLQMNSLKPEDTAVYFCAVCYNLETYWGQGTQVTVSSGGGGSGGGGSGGGGSVQNESKRYTVSYLKTLNYYDLVDLLVKTEIENLPDLFQYSSDAKEFYGNKTRMSFIMDEIGRRAPQYTEIDHKGIPTLVEVVRAGFYLGFHNKELNEINKRSFKERVIPSILAIQKNPNFKLGTEVQDKIVSATGLLAGNETAPPEVVNNFTPILQDCIKNIDRYALDDLKSKALFNVLAAPTYDITEYLRATKEKPENTPWYGKIDGFINELKKLALYGKINDNNSWIIDNGIYHIAPLGKLHSNNKIGIETLTEVMKVYPYLSMQHLQSADQIKRHYDSKDAEGNKIPLDKFKKEGKEKYCPKTYTFDDGKVIIKAGARVEEEKVKRLYWASKEVNSQFFRVYGIDKPLEEGNPDDILTMVIYNSPEEYKLNSVLYGYDTNNGGMYIEPEGTFFTYEREAQESTYTLEELFRHEYTHYLQGRYAVPGQWGRTKLYDNDRLTWYEEGGAELFAGSTRTSGILPRKSIVSNIHNTTRNNRYKLSDTVHSKYGASFEFYNYACMFMDYMYNKDMGILNKLNDLAKNNDVDGYDNYIRDLSSNYALNDKYQDHMQERIDNYENLTVPFVADDYLVRHAYKNPNEIYSEISEVAKLKDAKSEVKKSQYFSTFTLRGSYTGGASKGKLEDQKAMNKFIDDSLKKLDTYSWSGYKTLTAYFTNYKVDSSNRVTYDVVFHGYLPNEGDSKNSLPYGKINGTYKGTEKEKIKFSSEGSFDPDGKIVSYEWDFGDGNKSNEENPEHSYDKVGTYTVKLKVTDDKGESSVSTTTAEIKDLSENKLPVIYMHVPKSGALNQKVVFYGKGTYDPDGSIAGYQWDFGDGSDFSSEQNPSHVYTKKGEYTVTLRVMDSSGQMSEKTMKIKITDPVYPIGTEKEPNNSKETASGPIVPGIPVSGTIENTSDQDYFYFDVITPGEVKIDINKLGYGGATWVVYDENNNAVSYATDDGQNLSGKFKADKPGRYYIHLYMFNGSYMPYRINIELEHHHHHH

SEQ ID NO: 2 - DNA, 3336 bp, Artificial Sequence
  nucleic acid sequence encoding fusion protein
catatgcaggttcagctgcaagaaagcggtggtggtagcgttcaggcaggcggtagcctgaaactgacctgtgcagcaagcggttatatctttaatagctgtggtatgggttggtatcgtcagagtccgggtcgtgaacgtgaactggttagccgtattagcggtgatggtgatacctggcataaagaaagcgttaaaggtcgttttaccatcagccaggataacgtgaaaaaaaccctgtacctgcagatgaatagtctgaaaccggaagataccgcagtgtatttttgtgccgtttgctataatctggaaacctattggggtcagggcacccaggttaccgttagctcaggtggtggtggcagcggtggcggtggttctggtggcggaggtagcgtgcagaatgaaagcaaacgttataccgtgagctatctgaaaaccctgaactattatgatctggttgatctgctggtgaaaaccgaaattgaaaatctgccggacctgtttcagtatagcagtgatgcaaaagaattctacggtaataaaacccgcatgagctttatcatggatgaaattggtcgtcgtgcaccgcagtatacagaaattgatcataaaggtattccgacgctggttgaagttgttcgtgcaggtttttatctgggctttcataacaaagaactgaacgagattaacaaacgcagctttaaagaacgtgtgattccgagcattctggccattcagaaaaatccgaactttaaactgggcaccgaagtgcaggataaaattgttagcgcaaccggtctgctggcgggtaacgagaccgcgccgccggaagtggttaacaactttaccccgattctgcaggactgcattaaaaacattgaccgttatgcgctggatgacctgaagagcaaagcgctgtttaacgttctggcggcgccgacctatgacattaccgagtatctgcgtgcgaccaaggagaaaccggaaaacaccccgtggtacggcaaaatcgatggtttcattaacgagctgaaaaagctggcgctgtacggtaaaatcaacgacaacaacagctggatcattgacaacggtatttaccacatcgcgccgctgggcaaactgcacagcaacaacaagatcggcattgagaccctgaccgaagttatgaaggtgtacccgtatctgagcatgcaacacctgcagagcgcggatcaaatcaaacgtcactacgatagcaaggacgcggaaggcaacaaaatcccgctggacaaattcaagaaagaaggcaaggagaaatactgcccgaaaacctatacctttgatgacggcaaggttattatcaaggcgggtgcgcgtgtggaagaagagaaggtgaaacgtctgtattgggcgagcaaggaagtgaacagccagttctttcgtgtttatggcattgataaaccgctggaggaaggtaacccggatgacatcctgaccatggtgatctacaacagcccggaagagtacaaactgaacagcgtgctgtacggctacgacaccaacaacggtggcatgtacattgagccggaaggtacctttttcacctatgaacgtgaggcgcaggagagcacctataccctggaggagctgttccgtcacgagtatacccactatctgcaaggtcgttatgcggtgccgggccagtggggtcgtaccaaactgtacgataacgaccgtctgacctggtatgaggaaggcggtgcggagctgttcgcgggtagcacccgtaccagcggtattctgccgcgtaagagcatcgttagcaacattcacaacaccacccgtaacaaccgttacaagctgagcgacaccgtgcacagcaagtatggcgcgagcttcgaattctacaactacgcgtgcatgttcatggactatatgtacaacaaggacatgggcattctgaacaaactgaacgacctggcgaagaacaacgatgttgacggttacgacaactacattcgtgatctgagcagcaactatgcgctgaacgacaagtatcaggaccacatgcaggagcgtattgacaactacgagaacctgaccgttccgtttgttgcggacgattacctggttcgtcacgcgtacaagaacccgaacgaaatttatagcgaaatcagcgaggtggcgaaactgaaggatgcgaaaagcgaggttaaaaagagccaatacttcagcaccttcaccctgcgtggtagctataccggcggcgcgagcaagggcaaactggaggaccagaaagcgatgaacaagttcatcgacgatagcctgaagaagctggacacctatagctggagcggttacaaaaccctgaccgcgtacttcaccaactataaagttgacagcagcaaccgtgtgacctatgacgttgtgtttcacggttatctgccgaacgaaggcgatagcaagaacagcctgccgtatggtaaaatcaacggcacctacaagggtaccgaaaaggagaaaatcaagtttagcagcgaaggtagcttcgacccggatggcaaaatcgtgagctacgaatgggattttggtgacggtaacaaaagcaacgaagaaaacccggaacacagctacgataaagttggtacctacaccgtgaaactgaaagtgaccgatgacaagggcgaaagcagcgttagcaccaccaccgcggagatcaaagatctgagcgagaacaaactgccggtgatctacatgcacgttccgaaaagcggtgcgctgaaccagaaagttgtgttctatggcaaaggcacctacgatccggacggtagcatcgcgggctaccagtgggacttcggcgatggcagcgattttagcagcgagcagaacccgagccacgtgtataccaagaaaggcgaatataccgtgaccctgcgtgttatggacagcagcggtcagatgagcgagaagaccatgaaaatcaagattaccgatccggtttacccgattggtaccgagaaggaaccgaacaacagcaaggaaaccgcgagcggtccgattgtgccgggtattccggttagcggcaccatcgagaacaccagcgaccaagattatttttacttcgatgttatcaccccgggcgaggtgaagatcgatattaacaaactgggttacggttacggtggcgcgacctgggtggtttatgacgagaacaacaacgcggttagctacgcgaccgacgatggccagaacctgagcggcaagtttaaagcggataagccgggccgttactacatccacctgtatatgtttaacggtagctacatgccgtaccgtatcaacattgagctcgag

SEQ ID NO: 3 - protein, 115 aa, Artificial Sequence
  sdAb
QVQLQESGGGSVQAGGSLKLTCAASGYIFNSCGMGWYRQSPGRERELVSRISGDGDTWHKESVKGRFTISQDNVKKTLYLQMNSLKPEDTAVYFCAVCYNLETYWGQGTQVTVSS

SEQ ID NO: 4 - protein, 117 aa, Artificial Sequence
  sdAb
QVQLQESGGGLVQPGGSLRLSCAASGFIFSNDAMTWVRQAPGKGLEWVSSINWSGTHTNYADSVKGRFTISRDNAKRTLYLQMNSLKDEDTALYYCVTGYGVTKTPTGQGTQVTVSS

SEQ ID NO: 5 - protein, 912 aa, Clostridium histolyticum
VQNESKRYTVSYLKTLNYYDLVDLLVKTEIENLPDLFQYSSDAKEFYGNKTRMSFIMDEIGRRAPQYTEIDHKGIPTLVEVVRAGFYLGFHNKELNEINKRSFKERVIPSILAIQKNPNFKLGTEVQDKIVSATGLLAGNETAPPEVVNNFTPILQDCIKNIDRYALDDLKSKALFNVLAAPTYDITEYLRATKEKPENTPWYGKIDGFINELKKLALYGKINDNNSWIIDNGIYHIAPLGKLHSNNKIGIETLTEVMKVYPYLSMQHLQSADQIKRHYDSKDAEGNKIPLDKFKKEGKEKYCPKTYTFDDGKVIIKAGARVEEEKVKRLYWASKEVNSQFFRVYGIDKPLEEGNPDDILTMVIYNSPEEYKLNSVLYGYDTNNGGMYIEPEGTFFTYEREAQESTYTLEELFRHEYTHYLQGRYAVPGQWGRTKLYDNDRLTWYEEGGAELFAGSTRTSGILPRKSIVSNIHNTTRNNRYKLSDTVHSKYGASFEFYNYACMFMDYMYNKDMGILNKLNDLAKNNDVDGYDNYIRDLSSNYALNDKYQDHMQERIDNYENLTVPFVADDYLVRHAYKNPNEIYSEISEVAKLKDAKSEVKKSQYFSTFTLRGSYTGGASKGKLEDQKAMNKFIDDSLKKLDTYSWSGYKTLTAYFTNYKVDSSNRVTYDVVFHGYLPNEGDSKNSLPYGKINGTYKGTEKEKIKFSSEGSFDPDGKIVSYEWDFGDGNKSNEENPEHSYDKVGTYTVKLKVTDDKGESSVSTTTAEIKDLSENKLPVIYMHVPKSGALNQKVVFYGKGTYDPDGSIAGYQWDFGDGSDFSSEQNPSHVYTKKGEYTVTLRVMDSSGQMSEKTMKIKITDPVYPIGTEKEPNNSKETASGPIVPGIPVSGTIENTSDQDYFYFDVITPGEVK

SEQ ID NO: 6 - protein, 12 aa, Artificial Sequence
  linker
GGGSGGGSGGGS

SEQ ID NO: 7 - protein, 20 aa, Artificial Sequence
  linker
GGGSGGGSLEVLFQGPGGGS

SEQ ID NO: 8 - protein, 46 aa, Artificial Sequence
  ABD035
LAEAKVLANRELDKYGVSDFYKRLINKAKTVEGVEALKLHILAALP

SEQ ID NO: 9 - protein, 1175 aa, Artificial Sequence
  fusion protein
QVQLQESGGGSVQAGGSLKLTCAASGYIFNSCGMGWYRQSPGRERELVSRISGDGDTWHKESVKGRFTISQDNVKKTLYLQMNSLKPEDTAVYFCAVCYNLETYWGQGTQVTVSSGGGSGGGSGGGSVQNESKRYTVSYLKTLNYYDLVDLLVKTEIENLPDLFQYSSDAKEFYGNKTRMSFIMDEIGRRAPQYTEIDHKGIPTLVEVVRAGFYLGFHNKELNEINKRSFKERVIPSILAIQKNPNFKLGTEVQDKIVSATGLLAGNETAPPEVVNNFTPILQDCIKNIDRYALDDLKSKALFNVLAAPTYDITEYLRATKEKPENTPWYGKIDGFINELKKLALYGKINDNNSWIIDNGIYHIAPLGKLHSNNKIGIETLTEVMKVYPYLSMQHLQSADQIKRHYDSKDAEGNKIPLDKFKKEGKEKYCPKTYTFDDGKVIIKAGARVEEEKVKRLYWASKEVNSQFFRVYGIDKPLEEGNPDDILTMVIYNSPEEYKLNSVLYGYDTNNGGMYIEPEGTFFTYEREAQESTYTLEELFRHEYTHYLQGRYAVPGQWGRTKLYDNDRLTWYEEGGAELFAGSTRTSGILPRKSIVSNIHNTTRNNRYKLSDTVHSKYGASFEFYNYACMFMDYMYNKDMGILNKLNDLAKNNDVDGYDNYIRDLSSNYALNDKYQDHMQERIDNYENLTVPFVADDYLVRHAYKNPNEIYSEISEVAKLKDAKSEVKKSQYFSTFTLRGSYTGGASKGKLEDQKAMNKFIDDSLKKLDTYSWSGYKTLTAYFTNYKVDSSNRVTYDVVFHGYLPNEGDSKNSLPYGKINGTYKGTEKEKIKFSSEGSFDPDGKIVSYEWDFGDGNKSNEENPEHSYDKVGTYTVKLKVTDDKGESSVSTTTAEIKDLSENKLPVIYMHVPKSGALNQKVVFYGKGTYDPDGSIAGYQWDFGDGSDFSSEQNPSHVYTKKGEYTVTLRVMDSSGQMSEKTMKIKITDPVYPIGTEKEPNNSKETASGPIVPGIPVSGTIENTSDQDYFYFDVITPGEVKIDINKLGYGGATWVVYDENNNAVSYATDDGQNLSGKFKADKPGRYYIHLYMFNGSYMPYRINIEGGGSGGGSLEVLFQGPGGGSLAEAKVLANRELDKYGVSDFYKRLINKAKTVEGVEALKLHILAALPHHHHHH

SEQ ID NO: 10 - protein, 1177 aa, Artificial Sequence
  fusion protein
QVQLQESGGGLVQPGGSLRLSCAASGFIFSNDAMTWVRQAPGKGLEWVSSINWSGTHTNYADSVKGRFTISRDNAKRTLYLQMNSLKDEDTALYYCVTGYGVTKTPTGQGTQVTVSSGGGSGGGSGGGSVQNESKRYTVSYLKTLNYYDLVDLLVKTEIENLPDLFQYSSDAKEFYGNKTRMSFIMDEIGRRAPQYTEIDHKGIPTLVEVVRAGFYLGFHNKELNEINKRSFKERVIPSILAIQKNPNFKLGTEVQDKIVSATGLLAGNETAPPEVVNNFTPILQDCIKNIDRYALDDLKSKALFNVLAAPTYDITEYLRATKEKPENTPWYGKIDGFINELKKLALYGKINDNNSWIIDNGIYHIAPLGKLHSNNKIGIETLTEVMKVYPYLSMQHLQSADQIKRHYDSKDAEGNKIPLDKFKKEGKEKYCPKTYTFDDGKVIIKAGARVEEEKVKRLYWASKEVNSQFFRVYGIDKPLEEGNPDDILTMVIYNSPEEYKLNSVLYGYDTNNGGMYIEPEGTFFTYEREAQESTYTLEELFRHEYTHYLQGRYAVPGQWGRTKLYDNDRLTWYEEGGAELFAGSTRTSGILPRKSIVSNIHNTTRNNRYKLSDTVHSKYGASFEFYNYACMFMDYMYNKDMGILNKLNDLAKNNDVDGYDNYIRDLSSNYALNDKYQDHMQERIDNYENLTVPFVADDYLVRHAYKNPNEIYSEISEVAKLKDAKSEVKKSQYFSTFTLRGSYTGGASKGKLEDQKAMNKFIDDSLKKLDTYSWSGYKTLTAYFTNYKVDSSNRVTYDVVFHGYLPNEGDSKNSLPYGKINGTYKGTEKEKIKFSSEGSFDPDGKIVSYEWDFGDGNKSNEENPEHSYDKVGTYTVKLKVTDDKGESSVSTTTAEIKDLSENKLPVIYMHVPKSGALNQKVVFYGKGTYDPDGSIAGYQWDFGDGSDFSSEQNPSHVYTKKGEYTVTLRVMDSSGQMSEKTMKIKITDPVYPIGTEKEPNNSKETASGPIVPGIPVSGTIENTSDQDYFYFDVITPGEVKIDINKLGYGGATWVVYDENNNAVSYATDDGQNLSGKFKADKPGRYYIHLYMFNGSYMPYRINIEGGGSGGGSLEVLFQGPGGGSLAEAKVLANRELDKYGVSDFYKRLINKAKTVEGVEALKLHILAALPHHHHHH

(19) United States
(12) Patent Application Publication
(10) Pub. No.: US 2022/0363778 A1
(43) Pub. Date: Nov. 17, 2022

(54) TARGETED DELIVERY OF TUMOR MATRIX MODIFYING ENZYMES

(71) Applicant: The Research Foundation for The State University of New York, Amherst, NY (US)

(72) Inventors: Joseph P. BALTHASAR, Lancaster, NY (US); Brandon M. BORDEAU, Amherst, NY (US)

(21) Appl. No.: 17/638,725

(22) PCT Filed: Aug. 28, 2020
(86) PCT No.: PCT/US2020/048595

§ 371 Date: Feb. 25, 2022

Related U.S. Application Data
(60) Provisional application No. 62/893,120, filed on Aug. 28, 2019.

(51) Int. Cl. C07K 16/32; C12N 9/64; A61K 47/68; A61P 35/00

(52) U.S. Cl. CPC C07K 16/32; C12N 9/6489; A61K 47/6803; A61K 47/6855; A61P 35/00; C07K 2319/00; C07K 2317/569; A61K 2039/505

(57) ABSTRACT

Provided are compositions and methods for treatment of tumors. The compositions comprises a fusion construct comprising single domain antibody (sdAb) that is specific for HER2, collagenase, and optionally, albumin binding domain. Methods are provided for increasing penetrability of tumors and inhibiting the growth of tumors comprising administering a fusion construct comprising anti-HER2 specific sdAb, collagenase, and optionally albumin binding domain, alone or in combination with and an anti-tumor agent.

### CROSS-REFERENCE TO RELATED APPLICATIONS

This application claims priority to U.S. Provisional patent application no. 62/893,120, filed on Aug. 28, 2019, the disclosure of which is incorporated herein by reference.

### BACKGROUND OF THE DISCLOSURE

Dense composition of stroma in solid tumors can act as a barrier for intra-tumoral drug distribution. While administration of matrix-modulating enzymes, such as hyaluronidase and collagenase may reduce stromal density leading to decrease in intra-tumoral interstitial pressure and increase in distribution and efficacy of administered anti-cancer therapies (Dolor et al., Digesting a Path Forward: The Utility of Collagenase Tumor Treatment for Improved Drug Delivery. Mol Pharm. 2018; 15(6):2069-83; Magzoub et al., FASEB journal: official publication of the Federation of American Societies for Experimental Biology. 2008; 22(1):276-84), matrix-degrading enzymes are often associated with substantial systemic toxicity (Ramanathan et al., J Clin Oncol. 2019:JCO1801295. doi: 10.1200/JCO.18.01295. PubMed PMID: 30817250). Therefore, it has heretofore not been feasible to exploit the potential of matrix modulating enzymes in the treatment of cancer.

### SUMMARY OF THE DISCLOSURE

This disclosure provides compositions and methods for improving penetration of tumors by modified tumor-specific antibodies. The compositions comprise a fusion protein of a tumor specific antibody (or a tumor antigen binding fragment or derivative thereof) and a matrix modifying enzyme. In an embodiment, the antibody fragment is a monomeric antibody fragment that is specific for HER2+ tumors. In an embodiment, the matrix modifying enzyme is collagenase. In an embodiment, the antibody fragment is a sdAb that is specific for HER2 and the matrix modifying enzyme is collagenase.

This disclosure also provides a method for treatment of solid tumors comprising administering to an individual in need of treatment a composition comprising an antibody (or an antigen binding fragment or derivative thereof) fused to a matrix modifying enzyme. In an embodiment, the fusion protein is a monomeric antigen binding fragment of an HER2 specific antibody and collagenase.

In an embodiment, the fusion protein of an antibody or an antigen binding fragment or derivative thereof (such as a HER2 specific antibody) and matrix modifying enzyme (such as collagenase) is administered in combination with an anti-tumor agent such as an anti-tumor antibody, a derivative or fragment thereof (such as HER2 specific antibody), an antibody-drug conjugate or an anti-tumor macromolecule, whereby the distribution of the anti-tumor agent within the tumor is greater than if administered without the fusion protein. The fusion protein and the anti-tumor agent may be administered together or separately.

In an embodiment, this disclosure provides a fusion protein comprising collagenase and a single domain antibody (sdAb) which is specific for HER2. The sdAb may bind to an epitope of HER2 that is distinct from the epitope targeted by trastuzumab and/or the epitope targeted by pertuzumab. In an embodiment, the fusion protein further comprises an albumin binding domain. The fusion protein may comprise linkers between the collagenase, the sdAb and the albumin binding domain.

### BRIEF DESCRIPTION OF THE FIGURES

FIG. 1: Representation of the production, purification, characterization of anti-HER2-collagenase fusion proteins.

FIG. 2: Effects of targeted collagenase fusion protein on trastuzumab efficacy in mice bearing HER2+ tumors.

FIG. 3: Collagenase fusion protein structure and sequence. A schematic representation for three fusion proteins: 2Rs15d-ColH-ABD, 2Rb17c-ColH-ABD and 2Rs15d-ColH is shown. Anti-HER2 single domain antibodies are on the N-terminus and separated from collagenase with a (G4S)₃ linker. Collagenase is separated from the albumin-binding domain with a (G4S)₃ linker with an internal TEV protease cleavage site. All constructs were expressed with a hexahistidine tag for purification. Amino acid sequences for individual domains are 2Rs15d: SEQ ID NO:3, 2Rb17c: SEQ ID NO:4, Clostridium collagenase H: SEQ ID NO:5, Linker 1: SEQ ID NO:6, Linker 2: SEQ ID NO: 7 and ABD035: SEQ ID NO:8.

FIG. 4: Expression and SPR characterization of 2Rs15d- and 2Rb17c-ColH-ABD constructs. Top Left: SDS-PAGE analysis of 2Rs15d-ColH-ABD following expression and purification. The gel order from left to right is total protein, soluble protein, nickel-colum flow-through, wash, elution 1, elution 2, elution 1 buffer exchanged. The purified construct is highlighted in the box. Top middle/right: Shown are the SPR sensorgrams of 2Rs15d-ColH-ABD binding to HER2-Fc and mouse serum albumin respectively. Bottom left: SDS-PAGE of 2Rb17c-ColH-ABD expression and purification. The gel order from left to right is total protein, soluble protein, soluble protein filtered, nickel-column flow-through, wash 1, wash 2, elution 1, elution 1 buffer exchanged, elution 2. The purified construct is highlighted in the box. Bottom middle/right: Shown are the observed SPR sensorgrams of 2Rb17c-ColH-ABD binding to HER2-Fc and mouse serum albumin respectively.

FIG. 5: Expression and SPR characterization of 2Rs15d-ColH. Left: SDS-PAGE of 2Rs15d-ColH expression and purification. The gel order from left to right is soluble protein, nickel-column flow through, elution, elution buffer exchanged. The purified construct is highlighted in the box. Middle: Shown is the sensorgram of 2Rs15d-ColH binding to HER2-Fc and best-fit rate constants. Right: Sensorgram of 2Rs15d-ColH and 2Rs15d-ColH-ABD following injection over a HER2-Fc chip and chased with mouse serum albumin. At the time of MSA injection (˜400 seconds), the binding signal for 2Rs15d-ColH-ABD increases while the 2Rs15d-ColH signal is unchanged, indicating the ABD domain was successfully removed.

FIG. 6: Plasma time profiles for 2Rs15d-ColH and 2Rs15d-ColH-ABD. Observed plasma time profiles for 2Rs15d-ColH and 2Rs15d-ColH-ABD are shown. 2Rs15d-ColH-ABD demonstrated higher plasma retention of enzyme activity in comparison to 2Rs15d-ColH. Points represent the mean of three mice with standard deviation error bars.

FIG. 7: 2Rs15d-ColH-ABD increases trastuzumab tumor uptake in NCI-N87 tumors. NCI-N87 xenograft bearing mice were administered AF680-trastuzumab with and without 2Rs15d-ColH-ABD. Trastuzumab uptake was assessed ex-vivo using fluorescence microscopy with blood vessels labeled using AF555-anti-CD31. Displayed in panel A is a representative region from a whole tumor section (5B) for the trastuzumab only group. Shown in panel C is a representative section from a whole tumor section (5D) for the trastuzumab/2Rs15d-ColH-ABD group. Co-administration of 2Rs15d-ColH-ABD led to a dramatic increase in the fluorescence intensity for trastuzumab and increased the vasculature staining for intra-tumoral vessels in comparison to the PBS control group.

FIG. 8: Impact of co-administered 2Rs15d-ColH-ABD on trastuzumab tumor uptake. Tumor sections were analyzed to quantitatively assess the impact of 2Rs15d-ColH-ABD on trastuzumab tumor disposition. The mean of three slices represents an individual tumor with 2 tumors per group. 2Rs15d-ColH-ABD significantly increased the mean fluorescent intensity and total tumor area that stained positive for trastuzumab (p<0.05).

FIG. 9: Impact of co-administered 2Rs15d-ColH-ABD on tumor collagen. NCI-N87 xenograft bearing mice were administered 2Rs15d-ColH-ABD or a PBS vehicle. Tumor collagen and blood vessels are shown. Panel A displays a representative section of a whole tumor slice (7B) taken from the PBS administered mouse. Shown in C is a representative section of a whole tumor slice (7D) obtained from the 2Rs15d-ColH-ABD administered mouse. Dense, organized collagen networks can be observed for the PBS control tumor while the collagen in the 2Rs15d-ColH-ABD administered tumor appears more disperse and thinner. Tumor vasculature for the control tumor is surrounded by collagen, with most vessels being collapsed. In the 2Rs15d-ColH-ABD administered tumor, there are several regions in which the perivascular collagen is decreased, and tumor vasculature radius is greater, in comparison to the saline treated tumor.

FIG. 10: T-DM1 efficacy with and without co-administration of ColH/2Rs15d-ColH/2Rs15d-ColH-ABD. Top Left: Tumor growth curves for each dose group with curves ending 14 days after dosing. Tumor volume data represents the group mean with standard deviation error bars. Top Right: Survival curves for each group. Bottom Left: Exponential growth rate constants fit to the observed tumor volume data up to day 4, with standard deviation error bars. Bottom Right: Observed mouse bodyweight for individual groups, with standard deviation error bars.

### DETAILED DESCRIPTION OF THE DISCLOSURE

The present disclosure provides fusion proteins comprising antibody fragments having improved tumor penetrability and/or facilitating penetrability of other anti-tumor agents. Compositions comprising such fusion proteins and methods of using same are also provided.

The term “treatment” as used herein refers to reduction or delay in one or more symptoms or features associated with the presence of the particular condition being treated Treatment does not necessarily mean complete cure and does not preclude relapse of the condition. Treatment may be carried out over a short period of time (days, weeks), or over a long period of time (months) or may be on a continuous basis (e.g., in the form of a maintenance therapy). Treatment may be continual or intermittent.

The term “therapeutically effective amount” as used herein is the amount sufficient to achieve, in a single or multiple doses, the intended purpose of treatment. The exact amount desired or required will vary depending on the mode of administration, patient specifics and the like. Appropriate effective amounts can be determined by one of ordinary skill in the art (such as a clinician) with the benefit of the present disclosure.

Where a range of values is provided in this disclosure, it should be understood that each intervening value, to the tenth of the value of the lower limit between the upper and lower limit of that range, and any other intervening value in that stated range is encompassed within the disclosure, unless clearly indicated otherwise. The upper and lower limits of these smaller ranges may independently be included in the smaller ranges encompassed within the disclosure.

As used in this disclosure, the singular forms include the plural forms and vice versa unless the context clearly indicates otherwise.

The term “single domain antibody” (sdAb) is used interchangeably with the term “nanobody” to mean an antibody fragment representing a single monomeric variable antibody domain which is able to bind selectively to an antigen. A sdAb may comprise heavy chain variable domains or light chain variable domains. In an embodiment, the sdAb of the disclosure comprises heavy chain variable domain. A sdAb or nanobody may be derived from camelids (VHH fragments) or cartilaginous fishes (VNAR fragments), or may be derived from splitting the dimeric variable domains from IgG into monomers.

A reference to antibody derivatives and fragments in this disclosure includes any antigen binding fragment of an antibody or modification of the antibody. Examples include, but are not limited to, Fab, F(ab′), F(ab′)2, Fv, dAb, Fd, CDR fragments, single-chain antibodies (scFv), bivalent single-chain antibodies, single-chain phage antibodies, diabodies, nanobodies, chimeric antibodies, and fusion proteins comprising any of the foregoing or comprising an antibody or ADC.

In an aspect, this disclosure provides a fusion protein (also referred to herein as a fusion construct) of collagenase and a single domain antibody that binds specifically to human epidermal growth factor receptor 2 (HER2). In an embodiment, the Kd value for the sdAb binding to HER2 may be less than 50 nM. In embodiments, the Kd may be less than 25, 10, 5, 1 nM, or less than 750, 500, or 100 pM. The collagenase may be from any source. In an embodiment, the collagenase is clostridial collagenase H. The sdAb may be a HER2 binding domain of an antibody that is specific for HER2. In an embodiment, the antibody binds to an epitope that is distinct from the epitope targeted by trastuzumab and therefore, does not compete with trastuzumab or TDM1 for HER2 binding. In an embodiment, the sdAb is 2Rs15d, a dromedary-derived sdAb first reported by Vaneycken et.al. 2011 (PMID: 21478264). In an embodiment, an anti-HER2 sdAb, 2Rb17c may be used instead of 2Rs15d.

The collagenase may be directly linked to the sdAb or indirectly linked to the sdAb via a linker. The fusion protein may comprise the C-terminal of collagenase linked directly to the N-terminal of the sdAb, or may comprise the C-terminal of the sdAb linked directly to the N-terminal of collagenase. In an embodiment, this disclosure provides a fusion protein comprising: collagenase, sdAb that binds specifically to HER2, and a linker linking the C-terminal of the sdAb to the N-terminal of the collagenase. In an embodiment, this disclosure provides a fusion protein comprising: collagenase, sdAb that binds specifically to HER2, and a linker linking the C-terminal of the collagenase to the N-terminal of the sdAb. Linking the sdAb to collagenase, either directly or via a linker, should not eliminate either the binding function of the sdAb or the enzymatic function of collagenase. Suitable linkers include amino acid chains and alkyl chains functionalized with reactive groups for coupling to both the nanobody and collagenase. An amino acid chain linker may be about 1 to about 40 amino acid residues, such as 1 to 10 amino acid residues. In an embodiment, the fusion protein may further comprise an albumin binding domain, which may be present at the N-terminal end, the C-terminal end, or between the sdAb and collagenase.

An advantage of fusing the single domain antibody to a matrix digesting enzyme is the tumor selectivity advantage provided through the sdAb that limits off-target exposure to active enzyme.

In an embodiment, the fusion protein of the present disclosure comprises, consists essentially of, or consists of the amino acid sequence of SEQ ID NO:1. In this fusion protein, amino acids 1 to 115 represent the sdAb (2Rs15d) and amino acids 131 to 976 represent collagenase (colH), and amino acids 116 to 130 represent the linker (glycine serine linker). A polyhistidine tag (hexahistidine tag) is also shown from amino acids 977 to 982. The disclosure also encompasses fusion proteins comprising amino acids 1 to 115 and 131 to 976 without the intervening linker or with a different linker. In an embodiment, this disclosure provides variants of the fusion protein, wherein a variant of the fusion protein is at least 85%, 90%, 91%, 92%, 93%, 94%, 95%, 96%, 97%, 98% or 99% identical to the sequence of SEQ ID NO:1. Any variant of the fusion protein of this disclosure should have the HER2 binding function as well as the collagenase function of the fused protein of SEQ ID NO:1.

In an embodiment, the fusion protein of the present disclosure has a sequence which comprises a sdAb that binds specifically to HER2, and collagenase, and optionally, a linker, wherein the sequence of the sdAb is at least 85%, 90%, 91%, 92%, 93%, 94%, 95%, 96%, 97%, 98% or 99% identical to the sequence of amino acids 1 to 115 of SEQ ID NO:1, and wherein if the linker is present between the sdAb and collagenase, sequences may be, from N- to C-terminus, sdAb-linker-collagenase, or collagenase-linker-sdAb. In an embodiment, the fusion protein of the present disclosure has a sequence which comprises a sdAb that binds specifically to HER2, optionally a linker, and collagenase, wherein the sequence of the collagenase is at least 85%, 90%, 91%, 92%, 93%, 94%, 95%, 96%, 97%, 98% or 99% identical to the sequence of amino acids 131 to 976 of SEQ ID NO:1, and wherein if the linker is present between the sdAb and collagenase, sequences may be, from N- to C-terminus, sdAb-linker-collagenase, or collagenase-linker-sdAb.

In an aspect, the disclosure provides a nucleic acid sequence encoding a fusion protein of SEQ ID NO:1 (or a variant thereof) as described herein. In an embodiment, the nucleic acid comprises, consists essentially of, or consists of a sequence set forth in SEQ ID NO:2. In embodiments, the sequence which encodes a fusion protein or a variant thereof, as described herein may have at least 80%, 85%, 90%, 91%, 92%, 93%, 94%, 95%, 96%, 97%, 98% or 99% identical sequence to SEQ ID NO:2.

The amino acid sequence of a fusion construct, termed herein as 2Rs15d-ColH is provided below as SEQ ID NO:1. In the sequence, the 2Rs15d (sdAb) sequence is not underlined, the glycine serine linker is italicized, the ColH (collagenase) sequence is underlined, and the hexahistidine tag is bolded.

(SEQ ID NO: 1)
QVQLQESGGGSVQAGGSLKLTCAASGYIFNSCGMGWYRQSPGRERELVSR
ISGDGDTWHKESVKGRFTISQDNVKKTLYLQMNSLKPEDTAVYFCAVCYN
LETYWGQGTQVTVSSGGGGSGGGGSGGGGSVQNESKRYTVSYLKTLNYYD
LVDLLVKTEIENLPDLFQYSSDAKEFYGNKTRMSFIMDEIGRRAPQYTEI
DHKGIPTLVEVVRAGFYLGFHNKELNEINKRSFKERVIPSILAIQKNPNF
KLGTEVQDKIVSATGLLAGNETAPPEVVNNFTPILQDCIKNIDRYALDDL
KSKALFNVLAAPTYDITEYLRATKEKPENTPWYGKIDGFINELKKLALYG
KINDNNSWIIDNGIYHIAPLGKLHSNNKIGIETLTEVMKVYPYLSMQHLQ
SADQIKRHYDSKDAEGNKIPLDKFKKEGKEKYCPKTYTFDDGKVIIKAGA
RVEEEKVKRLYWASKEVNSQFFRVYGIDKPLEEGNPDDILTMVIYNSPEE
YKLNSVLYGYDTNNGGMYIEPEGTFFTYEREAQESTYTLEELFRHEYTHY
LQGRYAVPGQWGRTKLYDNDRLTWYEEGGAELFAGSTRTSGILPRKSIVS
NIHNTTRNNRYKLSDTVHSKYGASFEFYNYACMFMDYMYNKDMGILNKLN
DLAKNNDVDGYDNYIRDLSSNYALNDKYQDHMQERIDNYENLTVPFVADD
YLVRHAYKNPNEIYSEISEVAKLKDAKSEVKKSQYFSTFTLRGSYTGGAS
KGKLEDQKAMNKFIDDSLKKLDTYSWSGYKTLTAYFTNYKVDSSNRVTYD
VVFHGYLPNEGDSKNSLPYGKINGTYKGTEKEKIKFSSEGSFDPDGKIVS
YEWDFGDGNKSNEENPEHSYDKVGTYTVKLKVTDDKGESSVSTTTAEIKD
LSENKLPVIYMHVPKSGALNQKVVFYGKGTYDPDGSIAGYQWDFGDGSDF
SSEQNPSHVYTKKGEYTVTLRVMDSSGQMSEKTMKIKITDPVYPIGTEKE
PNNSKETASGPIVPGIPVSGTIENTSDQDYFYFDVITPGEVKIDINKLGY
GGATWVVYDENNNAVSYATDDGQNLSGKFKADKPGRYYIHLYMFNGSYMP
YRINIELEHHHHHH.

In an embodiment, the fusion construct comprises collagenase, sdAb which is specific for HER2, and an albumin binding domain (ABD). The ABD, the sdAb, and the collagenase may be present in any configuration from the N- to the C-terminus. The construct may further comprise linkers between the sdAb, collagenase and ABD and amino acid sequences, such as polyhistines, may flank the N- or the C-terminal ends. For example, the configuration may be sdAb-linker-ColH-linker-ABD-hexahistidine, or ABD-linker-sdAb-linker-collagenase-hexahistidine.

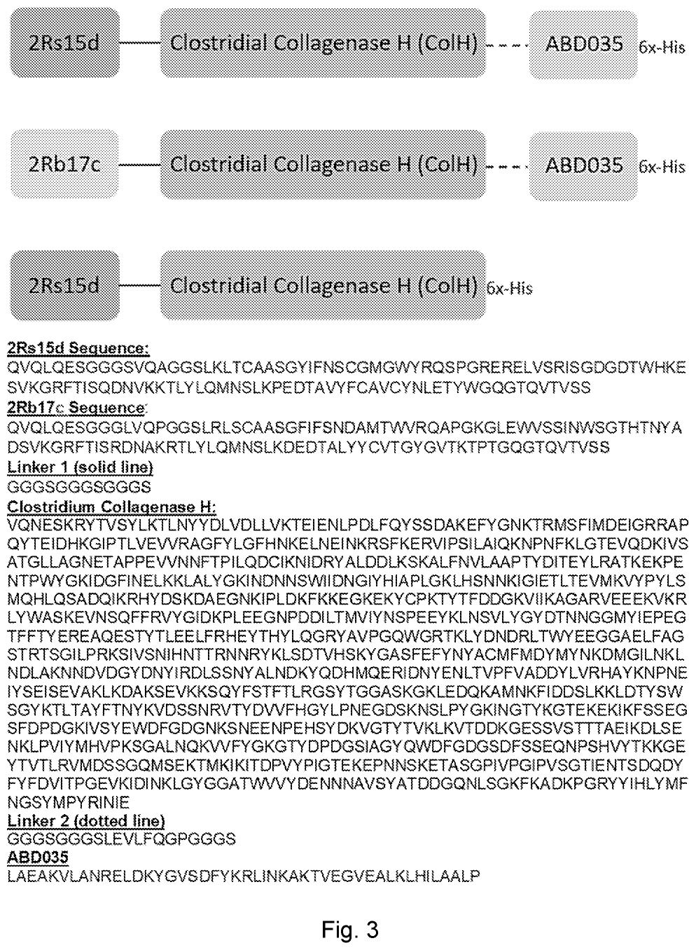

An example of a sequence of a fusion construct comprising sdAb 2Rs15d, collagenase and ABD is shown in FIG. 3 and the sequence is:

(SEQ ID NO: 9)
QVQLQESGGGSVQAGGSLKLTCAASGYIFNSCGMGWYRQSPGRERELVSR
ISGDGDTWHKESVKGRFTISQDNVKKTLYLQMNSLKPEDTAVYFCAVCYN
LETYWGQGTQVTVSSGGGSGGGSGGGSVQNESKRYTVSYLKTLNYYDLVD
LLVKTEIENLPDLFQYSSDAKEFYGNKTRMSFIMDEIGRRAPQYTEIDHK
GIPTLVEVVRAGFYLGFHNKELNEINKRSFKERVIPSILAIQKNPNFKLG
TEVQDKIVSATGLLAGNETAPPEVVNNFTPILQDCIKNIDRYALDDLKSK
ALFNVLAAPTYDITEYLRATKEKPENTPWYGKIDGFINELKKLALYGKIN
DNNSWIIDNGIYHIAPLGKLHSNNKIGIETLTEVMKVYPYLSMQHLQSAD
QIKRHYDSKDAEGNKIPLDKFKKEGKEKYCPKTYTFDDGKVIIKAGARVE
EEKVKRLYWASKEVNSQFFRVYGIDKPLEEGNPDDILTMVIYNSPEEYKL
NSVLYGYDTNNGGMYIEPEGTFFTYEREAQESTYTLEELFRHEYTHYLQG
RYAVPGQWGRTKLYDNDRLTWYEEGGAELFAGSTRTSGILPRKSIVSNIH
NTTRNNRYKLSDTVHSKYGASFEFYNYACMFMDYMYNKDMGILNKLNDLA
KNNDVDGYDNYIRDLSSNYALNDKYQDHMQERIDNYENLTVPFVADDYLV
RHAYKNPNEIYSEISEVAKLKDAKSEVKKSQYFSTFTLRGSYTGGASKGK
LEDQKAMNKFIDDSLKKLDTYSWSGYKTLTAYFTNYKVDSSNRVTYDVVF
HGYLPNEGDSKNSLPYGKINGTYKGTEKEKIKFSSEGSFDPDGKIVSYEW
DFGDGNKSNEENPEHSYDKVGTYTVKLKVTDDKGESSVSTTTAEIKDLSE
NKLPVIYMHVPKSGALNQKVVFYGKGTYDPDGSIAGYQWDFGDGSDFSSE
QNPSHVYTKKGEYTVTLRVMDSSGQMSEKTMKIKITDPVYPIGTEKEPNN
SKETASGPIVPGIPVSGTIENTSDQDYFYFDVITPGEVKIDINKLGYGGA
TWVVYDENNNAVSYATDDGQNLSGKFKADKPGRYYIHLYMFNGSYMPYRI
NIEGGGSGGGSLEVLFQGPGGGSLAEAKVLANRELDKYGVSDFYKRLINK
AKTVEGVEALKLHILAALPHHHHHH.

An example of a sequence of a fusion construct comprising sdAb 2Rb 17c, collagenase and ABD is shown in FIG. 3 and the sequence is:

(SEQ ID NO: 10)
QVQLQESGGGLVQPGGSLRLSCAASGFIFSNDAMTWVRQAPGKGLEWVSS
INWSGTHTNYADSVKGRFTISRDNAKRTLYLQMNSLKDEDTALYYCVTGY
GVTKTPTGQGTQVTVSSGGGSGGGSGGGSVQNESKRYTVSYLKTLNYYDL
VDLLVKTEIENLPDLFQYSSDAKEFYGNKTRMSFIMDEIGRRAPQYTEID
HKGIPTLVEVVRAGFYLGFHNKELNEINKRSFKERVIPSILAIQKNPNFK
LGTEVQDKIVSATGLLAGNETAPPEVVNNFTPILQDCIKNIDRYALDDLK
SKALFNVLAAPTYDITEYLRATKEKPENTPWYGKIDGFINELKKLALYGK
INDNNSWIIDNGIYHIAPLGKLHSNNKIGIETLTEVMKVYPYLSMQHLQS
ADQIKRHYDSKDAEGNKIPLDKFKKEGKEKYCPKTYTFDDGKVIIKAGAR
VEEEKVKRLYWASKEVNSQFFRVYGIDKPLEEGNPDDILTMVIYNSPEEY
KLNSVLYGYDTNNGGMYIEPEGTFFTYEREAQESTYTLEELFRHEYTHYL
QGRYAVPGQWGRTKLYDNDRLTWYEEGGAELFAGSTRTSGILPRKSIVSN
IHNTTRNNRYKLSDTVHSKYGASFEFYNYACMFMDYMYNKDMGILNKLND
LAKNNDVDGYDNYIRDLSSNYALNDKYQDHMQERIDNYENLTVPFVADDY
LVRHAYKNPNEIYSEISEVAKLKDAKSEVKKSQYFSTFTLRGSYTGGASK
GKLEDQKAMNKFIDDSLKKLDTYSWSGYKTLTAYFTNYKVDSSNRVTYDV
VFHGYLPNEGDSKNSLPYGKINGTYKGTEKEKIKFSSEGSFDPDGKIVSY
EWDFGDGNKSNEENPEHSYDKVGTYTVKLKVTDDKGESSVSTTTAEIKDL
SENKLPVIYMHVPKSGALNQKVVFYGKGTYDPDGSIAGYQWDFGDGSDFS
SEQNPSHVYTKKGEYTVTLRVMDSSGQMSEKTMKIKITDPVYPIGTEKEP
NNSKETASGPIVPGIPVSGTIENTSDQDYFYFDVITPGEVKIDINKLGYG
GATWVVYDENNNAVSYATDDGQNLSGKFKADKPGRYYIHLYMFNGSYMPY
RINIEGGGSGGGSLEVLFQGPGGGSLAEAKVLANRELDKYGVSDFYKRLI
NKAKTVEGVEALKLHILAALPHHHHHH.

In an embodiment, this disclosure provides variants of the fusion protein, wherein a variant of the fusion protein is at least 85%, 90%, 91%, 92%, 93%, 94%, 95%, 96%, 97%, 98% or 99% identical to the sequence of SEQ ID NO:9 or SEQ ID NO:10. Any variant of the fusion protein of this disclosure should have the HER2 binding function as well as the collagenase function.

An example of a nucleic acid sequence encoding a fusion construct of SEQ ID NO:1 is provided as SEQ ID NO:2, in which restriction enzyme site sequences are shown as bold and underlined, sequence encoding the 2Rs15d (sdAb) is neither underlined nor bolded, sequence encoding the glycine serine linker is italicized, and sequence encoding the ColH (collagenase) is underlined.

(SEQ ID NO: 2)
catatgcaggttcagctgcaagaaagcggtggtggtagcgttcaggcagg
cggtagcctgaaactgacctgtgcagcaagcggttatatctttaatagct
gtggtatgggttggtatcgtcagagtccgggtcgtgaacgtgaactggtt
agccgtattagcggtgatggtgatacctggcataaagaaagcgttaaagg
tcgttttaccatcagccaggataacgtgaaaaaaaccctgtacctgcaga
tgaatagtctgaaaccggaagataccgcagtgtatttttgtgccgtttgc
tataatctggaaacctattggggtcagggcacccaggttaccgttagctc
aggtggtggtggcagcggtggcggtggttctggtggcggaggtagcgtgc
agaatgaaagcaaacgttataccgtgagctatctgaaaaccctgaactat
tatgatctggttgatctgctggtgaaaaccgaaattgaaaatctgccgga
cctgtttcagtatagcagtgatgcaaaagaattctacggtaataaaaccc
gcatgagctttatcatggatgaaattggtcgtcgtgcaccgcagtataca
gaaattgatcataaaggtattccgacgctggttgaagttgttcgtgcagg
tttttatctgggctttcataacaaagaactgaacgagattaacaaacgca
gctttaaagaacgtgtgattccgagcattctggccattcagaaaaatccg
aactttaaactgggcaccgaagtgcaggataaaattgttagcgcaaccgg
tctgctggcgggtaacgagaccgcgccgccggaagtggttaacaacttta
ccccgattctgcaggactgcattaaaaacattgaccgttatgcgctggat
gacctgaagagcaaagcgctgtttaacgttctggcggcgccgacctatga
cattaccgagtatctgcgtgcgaccaaggagaaaccggaaaacaccccgt
ggtacggcaaaatcgatggtttcattaacgagctgaaaaagctggcgctg
tacggtaaaatcaacgacaacaacagctggatcattgacaacggtattta
ccacatcgcgccgctgggcaaactgcacagcaacaacaagatcggcattg
agaccctgaccgaagttatgaaggtgtacccgtatctgagcatgcaacac
ctgcagagcgcggatcaaatcaaacgtcactacgatagcaaggacgcgga
aggcaacaaaatcccgctggacaaattcaagaaagaaggcaaggagaaat
actgcccgaaaacctatacctttgatgacggcaaggttattatcaaggcg
ggtgcgcgtgtggaagaagagaaggtgaaacgtctgtattgggcgagcaa
ggaagtgaacagccagttctttcgtgtttatggcattgataaaccgctgg
aggaaggtaacccggatgacatcctgaccatggtgatctacaacagcccg
gaagagtacaaactgaacagcgtgctgtacggctacgacaccaacaacgg
tggcatgtacattgagccggaaggtacctttttcacctatgaacgtgagg
cgcaggagagcacctataccctggaggagctgttccgtcacgagtatacc
cactatctgcaaggtcgttatgcggtgccgggccagtggggtcgtaccaa
actgtacgataacgaccgtctgacctggtatgaggaaggcggtgcggagc
tgttcgcgggtagcacccgtaccagcggtattctgccgcgtaagagcatc
gttagcaacattcacaacaccacccgtaacaaccgttacaagctgagcga
caccgtgcacagcaagtatggcgcgagcttcgaattctacaactacgcgt
gcatgttcatggactatatgtacaacaaggacatgggcattctgaacaaa
ctgaacgacctggcgaagaacaacgatgttgacggttacgacaactacat
tcgtgatctgagcagcaactatgcgctgaacgacaagtatcaggaccaca
tgcaggagcgtattgacaactacgagaacctgaccgttccgtttgttgcg
gacgattacctggttcgtcacgcgtacaagaacccgaacgaaatttatag
cgaaatcagcgaggtggcgaaactgaaggatgcgaaaagcgaggttaaaa
agagccaatacttcagcaccttcaccctgcgtggtagctataccggcggc
gcgagcaagggcaaactggaggaccagaaagcgatgaacaagttcatcga
cgatagcctgaagaagctggacacctatagctggagcggttacaaaaccc
tgaccgcgtacttcaccaactataaagttgacagcagcaaccgtgtgacc
tatgacgttgtgtttcacggttatctgccgaacgaaggcgatagcaagaa
cagcctgccgtatggtaaaatcaacggcacctacaagggtaccgaaaagg
agaaaatcaagtttagcagcgaaggtagcttcgacccggatggcaaaatc
gtgagctacgaatgggattttggtgacggtaacaaaagcaacgaagaaaa
cccggaacacagctacgataaagttggtacctacaccgtgaaactgaaag
tgaccgatgacaagggcgaaagcagcgttagcaccaccaccgcggagatc
aaagatctgagcgagaacaaactgccggtgatctacatgcacgttccgaa
aagcggtgcgctgaaccagaaagttgtgttctatggcaaaggcacctacg
atccggacggtagcatcgcgggctaccagtgggacttcggcgatggcagc
gattttagcagcgagcagaacccgagccacgtgtataccaagaaaggcga
atataccgtgaccctgcgtgttatggacagcagcggtcagatgagcgaga
agaccatgaaaatcaagattaccgatccggtttacccgattggtaccgag
aaggaaccgaacaacagcaaggaaaccgcgagcggtccgattgtgccggg
tattccggttagcggcaccatcgagaacaccagcgaccaagattattttt
acttcgatgttatcaccccgggcgaggtgaagatcgatattaacaaactg
ggttacggttacggtggcgcgacctgggtggtttatgacgagaacaacaa
cgcggttagctacgcgaccgacgatggccagaacctgagcggcaagttta
aagcggataagccgggccgttactacatccacctgtatatgtttaacggt
agctacatgccgtaccgtatcaacattgagctcgag.

The amino acid sequence of sdAb 2Rs15d is also provided:

(SEQ ID NO: 3)
QVQLQESGGGSVQAGGSLKLTCAASGYIFNSCGMGWYRQSPGRERELVSR
ISGDGDTWHKESVKGRFTISQDNVKKTLYLQMNSLKPEDTAVYFCAVCYN
LETYWGQGTQVTVSS.

The amino acid sequence of another anti-HER2 sdAb 2Rb17c is:

(SEQ ID NO: 4)
QVQLQESGGGLVQPGGSLRLSCAASGFIFSNDAMTWVRQAPGKGLEWVSS
INWSGTHTNYADSVKGRFTISRDNAKRTLYLQMNSLKDEDTALYYCVTGY
GVTKTPTGQGTQVTVSS:

The amino acid sequence of Clostridium collagenase H is:

(SEQ ID NO: 5)
VQNESKRYTVSYLKTLNYYDLVDLLVKTEIENLPDLFQYSSDAKEFYGNK
TRMSFIMDEIGRRAPQYTEIDHKGIPTLVEVVRAGFYLGFHNKELNEINK
RSFKERVIPSILAIQKNPNFKLGTEVQDKIVSATGLLAGNETAPPEVVNN
FTPILQDCIKNIDRYALDDLKSKALFNVLAAPTYDITEYLRATKEKPENT
PWYGKIDGFINELKKLALYGKINDNNSWIIDNGIYHIAPLGKLHSNNKIG
IETLTEVMKVYPYLSMQHLQSADQIKRHYDSKDAEGNKIPLDKFKKEGKE
KYCPKTYTFDDGKVIIKAGARVEEEKVKRLYWASKEVNSQFFRVYGIDKP
LEEGNPDDILTMVIYNSPEEYKLNSVLYGYDTNNGGMYIEPEGTFFTYER
EAQESTYTLEELFRHEYTHYLQGRYAVPGQWGRTKLYDNDRLTWYEEGGA
ELFAGSTRTSGILPRKSIVSNIHNTTRNNRYKLSDTVHSKYGASFEFYNY
ACMFMDYMYNKDMGILNKLNDLAKNNDVDGYDNYIRDLSSNYALNDKYQD
HMQERIDNYENLTVPFVADDYLVRHAYKNPNEIYSEISEVAKLKDAKSEV
KKSQYFSTFTLRGSYTGGASKGKLEDQKAMNKFIDDSLKKLDTYSWSGYK
TLTAYFTNYKVDSSNRVTYDVVFHGYLPNEGDSKNSLPYGKINGTYKGTE
KEKIKFSSEGSFDPDGKIVSYEWDFGDGNKSNEENPEHSYDKVGTYTVKL
KVTDDKGESSVSTTTAEIKDLSENKLPVIYMHVPKSGALNQKVVFYGKGT
YDPDGSIAGYQWDFGDGSDFSSEQNPSHVYTKKGEYTVTLRVMDSSGQMS
EKTMKIKITDPVYPIGTEKEPNNSKETASGPIVPGIPVSGTIENTSDQDY
FYFDVITPGEVK.

The amino acid sequence for a glycine serine linker is:

(SEQ ID NO: 6)
GGGSGGGSGGGS.

The amino acid sequence for another linker is:

(SEQ ID NO: 7)
GGGSGGGSLEVLFQGPGGGS

The amino acid sequence for ABD035 is:

(SEQ ID NO: 8)
LAEAKVLANRELDKYGVSDFYKRLINKAKTVEGVEALKLHILAALP

The disclosure of a sequence in this disclosure with hexahistidines is intended to include a sequence of the construct without the hexahistidine also.

In an aspect, the present disclosure provides an expression vector comprising a sdAb-collagenase fusion construct of the disclosure. The expression vector is not particularly limiting other than by a requirement for the sdAb-collagenase fusion protein expression to be driven from a suitable promoter. Many suitable expression vectors and systems are commercially available. Examples of vectors include plasmids, cosmids, transposable elements, viruses (bacteriophage, animal viruses, and plant viruses), and artificial chromosomes (e.g., YACs). The expression vectors may be configured to produce fusion proteins. The fusion proteins may include components that facilitate purification, such as HIS or FLAG tag or improve solubility or secretion or other functions. The vector may have a high copy number, an intermediate copy number, or a low copy number. Expression vectors typically contain one or more of the following elements: promoters, terminators, ribosomal binding sites, and IRES. A promoter may comprise one or more specific transcriptional regulatory sequences to further enhance expression and/or to alter the spatial expression and/or temporal expression of a nucleic acid. A nucleic acid encoding a nanobody construct may also be operably linked to a nucleotide sequence encoding a selectable marker. A selectable marker may be used to efficiently select and identify cells that have integrated the exogenous nucleic acids. Selectable markers give the cell receiving the exogenous nucleic acid a selection advantage, such as resistance towards a certain toxin or antibiotic. Suitable examples of antibiotic resistance markers include those coding for proteins that impart resistance to kanamycin, streptomycin, spectinomycin, neomycin, gentamycin (G418), ampicillin, tetracycline, chloramphenicol, puromycin, hygromycin, zeocin, and blasticidin. An expression vector encoding a nanobody construct may be delivered to a host cell using a viral vector or via a non-viral method of transfer. Viral vectors suitable for introducing nucleic acids into cells include retroviruses, adenoviruses, adeno-associated viruses, rhabdoviruses, and herpes viruses. Non-viral methods of nucleic acid transfer include naked nucleic acid, liposomes, and protein/nucleic acid conjugates. An expression construct encoding a nanobody construct may be introduced into the cell by transfection. Appropriate host cells include, but are not limited to, bacterial, yeast, insect, and mammalian cells. In an embodiment, the expression system is a bacterial expression system, involving, for example, E. coli. Host cells may be transfected with a vector comprising a nanobody construct and then cultured so that they transcribe and translate the desired polypeptide. The host cells may then be lysed to extract the expressed polypeptide for subsequent purification.

In an aspect, this disclosure provides host cells containing vector constructs as described herein. Host cells may contain nucleotide sequences that are operably associated with one or more heterologous control regions (e.g., promoter and/or enhancer) using techniques known of in the art. The host cell can be a higher eukaryotic cell, such as a mammalian cell (e.g., a human derived cell), or a lower eukaryotic cell, such as a yeast cell, or the host cell can be a prokaryotic cell, such as a bacterial cell. A host strain may be such that it modulates the expression of the inserted gene sequences, or modifies and processes the gene product as desired. Expression from certain promoters may be modified by the presence of certain inducers thereby allowing the expression of the genetically engineered polypeptide to be controlled. In an embodiment, the disclosure provides a fusion protein of a sdAb which is specific for HER2 and a matrix modifying enzyme, and optionally albumin binding domain. The matrix modifying enzyme may be collagenase. In an embodiment, the sdAb binds to an epitope that is distinct from the epitope targeted by trastuzumab (Herceptin™) and, therefore, the sdAb would not compete with trastuzumab or TDM1 for HER2 binding. In another embodiment, the sdAb binds to an epitope that is distinct from the epitope targeted by pertuzumab (Perjeta). In an embodiment, the sdAb binds to an epitope that is distinct from the epitope targeted by either trastuzumab or pertuzumab.

In an aspect, the disclosure provides pharmaceutical compositions comprising the fusion protein as described herein. The formulations typically contain physiologically acceptable carriers, excipients or stabilizers and may be in the form of aqueous solutions, lyophilized or other dried or solid formulations. Examples of suitable pharmaceutical preparation components can be found in Remington: The Science and Practice of Pharmacy 20th edition (2000). Acceptable carriers, excipients, or stabilizers are nontoxic to recipients at the dosages and concentrations employed, and include buffers such as phosphate, citrate, histidine and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™, polyethylene glycol (PEG) and the like.

In an aspect, this disclosure provides a method for improving penetrability of an antitumor antigen antibody or a fragment thereof by administering to an individual in need of treatment a composition comprising the antibody or an antigen binding fragment thereof fused to a matrix modifying enzyme, such as collagenase, and optionally further comprising albumin binding domain. In an embodiment, the disclosure provides a method for inhibiting the growth of or proliferation of a tumor comprising administering to an individual in need of treatment anti-tumor agent, and an sdAb specific for a tumor antigen fused to a matrix modifying enzyme, such as collagenase, with the fusion protein optionally further comprising an albumin binding domain. In an embodiment, the disclosure provides a method for treatment of HER2+ tumors comprising administering to an individual who is afflicted with a HER2+ tumor, a composition comprising a HER2 specific sdAb fused to collagenase—either directly or via a linker, and optionally further comprising albumin binding domain in the fusion protein, whereby the penetrability of the fusion protein within the tumor is greater than the penetrability of the sdAb alone.

In an embodiment, the disclosure provides a method for improving the penetrability and distribution of an anti-tumor agent (such as an antibody, antibody derivative or fragment, antibody drug conjugate, anti-tumor macromolecule, anti-tumor molecule) within a tumor comprising administering to an individual in need of treatment the anti-tumor agent and a fusion protein comprising an antitumor antigen antibody or a fragment or derivative thereof and collagenase, and optionally an albumin binding domain. The anti-tumor agent and the anti-tumor antibody (or fragment or derivative thereof) may be the same or different. The anti-tumor agent and the fusion protein may be administered in the same composition or as separate compositions. When administered as separate compositions, they may be administered at the same time or different times, same route or different routes, over the same period of time or different periods of time, which may overlap. As an example, the disclosure provides a method for improving the distribution of trastuzumab or T-DM1 within a tumor comprising administering to the individual (and the tumor), the trastuzumab or T-DM1 and a fusion protein comprising anti-HER2 antibody or a fragment or derivative thereof, such as a sdAb (e.g., 2Rs15d), wherein the growth of the tumor is inhibited more than if the trastuzumab or T-DM1 is administered without the fusion protein. In an example, the disclosure provides a method for improving the distribution of pertuzumab within a tumor comprising administering to the individual (and the tumor), pertuzumab and a fusion protein comprising anti-HER2 antibody or a fragment or derivative thereof, such as a sdAb (e.g., 2Rs15d), wherein the growth of the tumor is inhibited more than if the pertuzumab is administered without the fusion protein.

In an embodiment, the fusion construct of the present disclosure improves the distribution of an anti-tumor agent that is administered in combination with the fusion construct. The anti-tumor agent may be an anti-tumor antigen antibody, an antibody derivative, antibody fragment or any macromolecule having anti-tumor growth activity. Antibody derivatives and fragments include, but are not limited to Fab, F(ab′), F(ab′)2, Fv, dAb, Fd, CDR fragments, single-chain antibodies (scFv), bivalent single-chain antibodies, single-chain phage antibodies, diabodies, nanobodies, chimeric antibodies, and fusion proteins comprising any of the foregoing or comprising an antibody or ADC.

In an embodiment, the disclosure provides a method for improving penetrability of an antitumor antibody or a conjugate of the antibody (such as an antibody-drug conjugate (ADC)) comprising administering to an individual in need of treatment, i) a fusion protein comprising a tumor antigen specific sdAb, tumor matrix modifying enzyme, and optionally, albumin binding domain, and ii) the antitumor antibody or a conjugate of the antibody (such as an ADC). In an embodiment, the method comprises administering to an individual in need of treatment, i) a fusion protein comprising a HER2 specific sdAb, collagenase, and optionally, an albumin binding domain; and ii) anti-HER2 antibody or an ADC comprising an anti-HER2 antibody. In an embodiment, the method comprises administering to an individual in need of treatment, i) a fusion protein comprising a HER2 specific sdAb (such 2Rs15d), collagenase, and optionally, an albumin binding domain; and ii) trastuzumab or T-DM1, or pertuzumab.

In an embodiment, the disclosure provides a method for inhibiting the growth of or proliferation of tumor cells by administering to an individual who is afflicted with the tumor a fusion protein comprising a sdAb that is specific for HER2, and collagenase, wherein the fusion protein penetrates further into the tumor than the sdAb without being fused to collagenase, and/or exhibits increased inhibition of tumor cell growth compared to sdAb without fusion to collagenase. In an embodiment, the fusion protein as administered in combination with an anti-tumor agent, such as an anti-tumor antibody, such as sdAb, wherein the fusion protein facilitates the penetration of accompanying anti-tumor agent, (e.g., sdAb).

A composition comprising the fusion protein may be administered using any suitable route including parenteral, subcutaneous, intraperitoneal, intrapulmonary, and intranasal, and, if desired for local treatment, intratumoral administration, or at or near the tumor. Parenteral infusions include intramuscular, intravenous, intraarterial, intraperitoneal, or subcutaneous administration. In an embodiment, the fusion protein is delivered intra-tumorally. The administration may be carried out in a continuous manner or may be intermittent. Appropriate dosage will depend upon the particular tumor being treated, the specifics and condition of the individual patient, the mode of administration etc. Determination of appropriate dosage is within the purview of one skilled in the art, such as a treating physician. In an embodiment, the amount of sdAb-collagenase fusion protein may be administered is from about 0.01 mg/kg to 500 mg/kg, or 0.1 mg/kg to about 100 mg/kg. In embodiments, the amount of fusion protein administered may be 0.1 mg/kg to about 50 mg/kg, or 0.1 mg/kg to about 25, 10, 5 or 1 mg/kg. In embodiments, the administered amount of the fusion protein may be 0.01, 0.05, 0.1, 0.5, 1.0, 5.0, 10.0, 25.0, 50.0, 75.0, 100.0, 200.0, or 500.0 mg/kg.

The fusion constructs and the antibody or sdAb or antibody conjugates (such as ADCs) may be administered in the same composition or in different compositions, at the same time or at different times, by the same route or different routes, over the same period of time or different periods of time.

The fusion constructs of the present disclosure, such as sdAb-collagenase-ABD fusion protein, may be administered alone or in combination with other types of treatments (e.g., surgical resection, radiation therapy, chemotherapy, hormonal therapy, immunotherapy or other anti-tumor agents). Similarly, the fusion constructs and the antibody or sdAb or antibody conjugates (such as ADCs) may be administered in combination with other types of treatments (e.g., surgical resection, radiation therapy, chemotherapy, hormonal therapy, immunotherapy or other anti-tumor agents).

The present compositions may be used for any type of cancer, including carcinoma, lymphoma, sarcoma, melanoma and leukemia. Non-limiting examples include squamous cell cancer, small-cell lung cancer, non-small cell lung cancer, adenocarcinoma of the lung, squamous carcinoma of the lung, cancer of the peritoneum, myeloma (including multiple myeloma), hepatocellular cancer, gastric cancer, intestinal cancer, pancreatic cancer, glioblastoma/glioma (e.g., anaplastic astrocytoma, glioblastoma multiforme, anaplastic oligodendroglioma, anaplastic oligodendroastrocytoma), cervical cancer, ovarian cancer, liver cancer, bladder cancer, hepatoma, breast cancer, brain cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancer. In an embodiment, the cancer is HER2+.In an embodiment, the tumor comprises dense collagen-containing extracellular matrix.

The present compositions may be particularly useful for patients where the HER2+ tumors, such as, for example, breast tumors, are found to be non-responsive to current treatments, such as trastuzumab, pertuzumab, and T-DM1. Alternatively, the present compositions may be used in combination with trastuzumab, pertuzumab, and/or T-DM1.

In an aspect, this disclosure provides kits for the treatment of cancer. The kit may comprise an anti-tumor agent and a fusion construct of the present disclosure. For example, a kit may comprise, in separate containers, trastuzumab, pertuzumab, and/or T-DM1, a fusion construct comprising sdAb directed to HER2, collagenase, and optionally albumin binding domain, and optionally instructions for use, which may include dosage and administration instructions. In an embodiment, the kit comprises in separate containers: i) trastuzumab, pertuzumab, and/or T-DM1, and 2) sdAb 2Rs15d. Multiple doses of the components may be provided.

The following are some non-restrictive examples of embodiments of the present disclosure.

Example 1. A fusion protein of a single domain antibody (sdAb) and collagenase.

Example 2. A fusion protein of a single domain antibody (sdAb) which is specific for HER2, and collagenase, wherein the sdAb and the collagenase are covalently linked.

Example 3. The fusion protein of Example 1 or Example 2, wherein the sdAb and the collagenase are covalently linked via a linker.

Example 4. A fusion protein having the sequence as set forth in SEQ ID NO:1.

Example 5. A pharmaceutical composition comprising the fusion protein of any one of Examples 1-4.

Example 6. A method of treating a tumor comprising administering to an individual in need of treatment a composition of Example 5.

Example 7. The method of Example 6, wherein the composition is delivered at or near a tumor or intratumorally.

The invention is further demonstrated by way of the figures and data presented herein.

### EXAMPLE 1

This example describes the development of anti-HER2-collagenase fusion proteins. 2Rs15d, a high affinity anti-HER2 sdAb, was employed as a model targeting vector. 2Rs15d binds to an epitope that is distinct from that targeted by trastuzumab and, consequently, 2Rs15d does not compete with trastuzumab or TDM1 for HER2 binding. Clostridium collagenase-H (ColH) was selected as a model matrix-modulating enzyme. 2Rs15d-ColH fusion proteins were expressed in E. coli and characterized for HER2 binding and for collagenase activity.

### Methods

### Development of 2Rs15d-Collagenase

DNA encoding for the 2Rs15d-Clostridial Collagenase H fusion protein (2Rs15d-ColH) was synthesized commercially by GenScript (SEQ ID NO:2). The DNA product was digested with XhoI and NdeI restriction enzymes and ligated into the Pet22b(+) plasmid. The E. coli strain SHuffle was transformed with the 2Rs15d-ColH Pet22b vector through heat shock and plated onto a LB agar plate with 100 μg/ml ampicillin and grown overnight at 30° C. Following incubation, a single transformed colony was picked with a sterile pipette tip and inoculated into 5 mL of LB medium and grown overnight at 30° C. in a shaker incubator (200 RPM, 18 hours). Following overnight growth, glycerol stocks were generated through a 1:1 dilution of the transformed SHuffle culture in 50% glycerol and stocks stored at −80° C. To express the 2Rs15d-ColH construct a glycerol stock was removed from the −80° C. and a small volume spread over a LB agar plate (100 μg/ml ampicillin) using a sterile inoculation loop. Following overnight incubation, a single colony was lifted from the LB agar plate and inoculated into a starter culture of LB medium with 100 μg/mL ampicillin in a shaker incubator (30° C., 200 RPM, 18 hours). Following an 18-hour incubation the starter culture was diluted 1/100× into LB medium containing 100 μg/ml ampicillin and grown in a shaker incubator at 30° C., 200 RPM. Cell density was monitored at OD 600 nm using a biospectrophotometer. Once the culture reached an optical density of 0.6-0.8, 2Rs15d-ColH expression was initiated through addition of 1 mM IPTG into the growth medium with expression proceeding for 18-20 hours at 14° C., 200 RPM. Following expression, E. coli cells were pelleted through centrifugation at 10,000×g for 5 minutes. Pelleted cells were lysed using Bugbuster® protein extraction reagent containing 1 mg/ml lysozyme and 0.25 units/mL Benzonase® Nuclease. The cell lysate, containing 2Rs15d-ColH, was passed over a 3 mL HisPur™ Ni-NTA spin column through gravity filtration to allow purification through the C-terminal hexahistidine tag on 2Rs15d-ColH, encoded as part of the Pet22b vector. Non-specifically bound protein was removed from the column using manufacturer recommendations for wash buffer composition and volume. Following washing, 2Rs15d-ColH was eluted using a 500 mM imidazole elution buffer. Following elution, the purified 2Rs15d-ColH was buffer exchanged into PBS using a 5 mL, 7 kDa molecular weight cut-off Zeba™ spin desalting column. The final purified product in PBS was analyzed for purity using SDS-PAGE.

### Characterization of 2Rs15d-ColH Enzyme Activity

A fluorescence-based plate assay was developed to determine the enzyme activity of purified 2Rs15d-ColH. The collagenase substrate fluorescein pig skin gelatin (1 mg per vial) was purchased from ThermoFisher (D12054) and diluted into distilled water at 1 mg/ml. 20 uL of the fluorescein substrate was added to individual wells of a 96 well Nunc MaxiSorp™ ELISA plate and diluted with 80 μl of the recommended activity buffer: 50 mM TRIS, 150 mM NaCl, 5 mM CaCl2 pH 7.6. A standard curve of collagenase activity was generated through serial dilution of a commercially obtained purified clostridium collagenase H (Worthington Biochemical, LS005273) with known activity between 0.05-5 units/mL in activity buffer. 100 uL of each ColH standard was added to the wells of the ELISA plate in duplicate in addition to serial dilutions of the unknown 2Rs15d-ColH and immediately placed into a SpectraMax i3 multi-mode microplate reader. Fluorescence was read every 30 seconds for 5 minutes at an excitation wavelength of 485 nm and an emission wavelength of 530 nm. A standard curve of enzyme activity was generated using the observed ΔFluorescence/minute for the ColH standards. The dilution of 2Rs15d-ColH that fell within the linear range of the standard curve was used to determine the number of enzyme units of the purified 2Rs15d-ColH product.

### Characterization of 2Rs15d-ColH HER2 Binding Affinity

Surface plasmon resonance was performed using a Reichert SR7500DC SPR and SR8100 autosampler to assess HER2 binding of the 2Rs15d-ColH product. HER2-Fc (Sino Biological, 10004-H02H) was immobilized onto a carboxymethyl dextran SPR chip (Reichert, 13206066) using EDC/NHS linker chemistry. Purified 2Rs15d-ColH was diluted into the SPR mobile phase (1×PBS, 0.005% Tween-20) at concentrations of 10, 25, 50, 100, 200 and 500 nM. Individual dilutions were injected over the HER2-Fc chip for 1 minute at a flow-rate of 25 μL per minute with a 60-minute dissociation at a flow rate of 25 uL per minute. The chip was regenerated between individual runs through injection of 10 mM Glycine pH 1.5 over the chip surface for 2 minutes at a flow rate of 25 uL per minute. The observed sensor-grams were fit to a 1:1 Langmuir binding model in the Scrubber analysis software to obtain kon, koff, and the equilibrium dissociation constant KD.

### Results

The effects of 2Rs15d-ColH on trastuzumab exposure and TDM1 efficacy in NCI-N87 tumors were evaluated. Initial investigations evaluated the effects of 50 units of untargeted collagenase and 50 units of targeted collagenase. (via the novel fusion protein, 2Rs15d-ColH) on trastuzumab distribution in mice bearing NCI-N87 tumors.

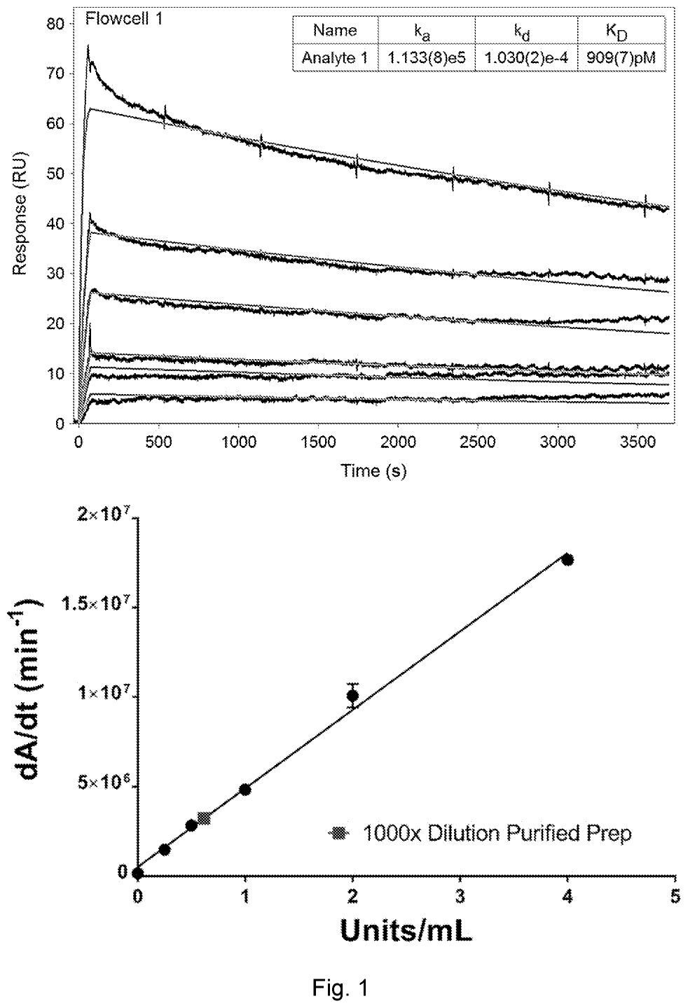

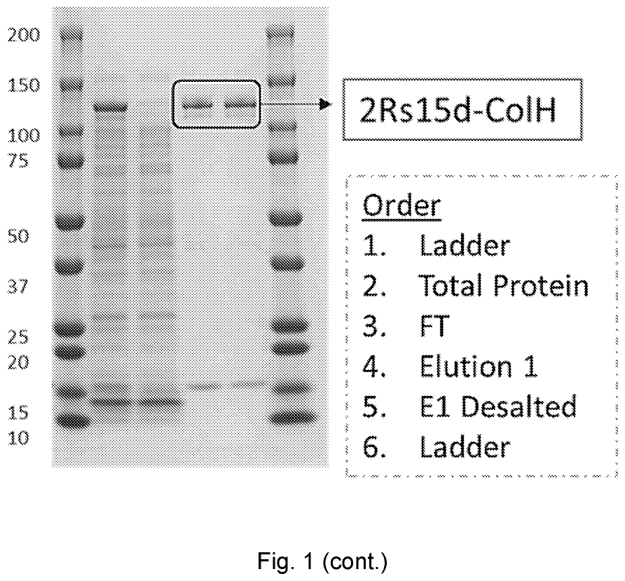

The production, purification, characterization of anti-HER2-collagenase fusion proteins was carried out. (FIG. 1). Novel fusion proteins combining an anti-HER2 sdAb and Clostridium collagenase H were expressed in E coli, and purified by affinity chromatography (SDS-PAGE, left-panel). Collagenase activity was assessed using a fluorescent collagenase substrate. A standard curve of Collagenase activity was generated using a purified commercial preparation of ColH with known enzyme activity. Serial dilutions of 2Rs15d between 100-10000x were assessed and activity determined using the standard curve (middle panel). The 2Rs15d-ColH fusion protein retained high affinity for HER2 (KD˜900 pM) (right panel).

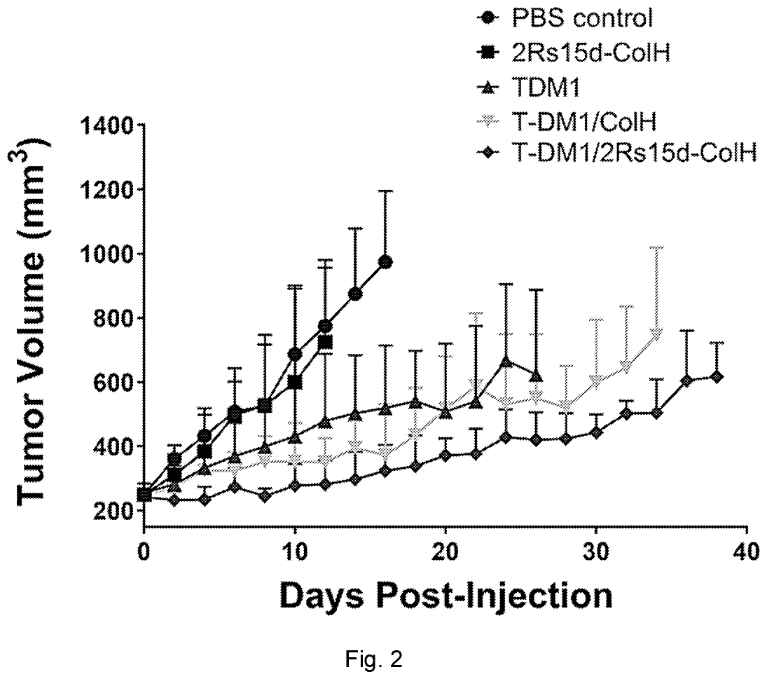

The effects of targeted collagenase fusion protein on trastuzumab efficacy in mice bearing HER2+ tumors is shown in FIG. 2. Mice bearing NCI-N87 tumors were treated with PBS (control), 2Rs15d-ColH (50 u), TDM1 (1.8 mg/kg), TDM1 and untargeted collagenase (1.8 mg/kg+50 u), or TDM1 and targeted collagenase (1.8 mg/kg+50u 2Rs15d-ColH). The combination of targeted collagenase and TDM1 led to superior anti-tumor activity. Note: The 1.8 mg/kg dose of TDM1 was selected for efficacy studies based on prior demonstrations of efficacy in the range of 1-3 mg/kg for treatment of NCI-N87 xenograft tumors.

### EXAMPLE 2

This example describes a fusion protein comprising ColH with an N-terminal anti-HER2 single domain antibody and a C-terminal albumin binding domain.

### Materials and Methods

Antibody/Cell-Lines/Mouse Models

Trastuzumab and T-DM1 were purchased from Millard Fillmore Memorial Hospital (Amherst, N.Y.). The gastric carcinoma cell-line NCI-N87 was cultured following American Type Culture Collection recommendations. Male Nu/J mice were purchased from The Jackson Laboratory (Bar Harbor, Me.), and male Swiss-Webster mice were purchased from Envigo (Indianapolis, Ind.). Nu/J mice were injected subcutaneously in the right flank with 100 μL of a 1:1 solution of matrigel (Thermo Fisher Scientific, Waltham, Mass., CB-40234):RPMI 1640 containing 5 million NCI-N87 cells for the T-DM1 efficacy study and with 200 μl of a 1:1 matrigel:RPMI 1640 solution containing 5 million NCI-N87 cells for the fluorescence studies.

### Fusion Protein Sequences

Examples of configurations for fusion construct comprising 2Rs15d, 2Rb17c, ColH and ABD035 are shown in FIG. 3. 2Rs15d or 2Rb17c was oriented on the amino(N)-terminus of ColH and separated from ColH using a (glycine₄serine)3 linker. ABD035 is oriented on the carboxy(C)-terminus and is separated from ColH by a (glycine₄serine)3 linker with an internal tobacco etch virus (TEV) protease cleavage site. DNA encoding for the fusion proteins was codon-optimized for E. coli expression and synthesized commercially by GenScript (Piscataway, N.J.). 2Rs15d-ColH was generated by restriction enzyme digestion of the 2Rs15d-ColH-ABD035 genetic sequence several hundred nucleotides upstream of the ABD sequence. ColH DNA that was removed by restriction enzyme digestion was replaced through ligation with synthesized DNA lacking the ABD sequence.

### Expression and Purification of Collagenase Fusion Proteins

DNA encoding for the fusion protein was digested with XhoI and NdeI restriction enzymes and ligated into the Pet22b(+) plasmid (Millipore-Sigma, Burlington, Mass., 69744). The E. coli strain SHuffle® (NEB, C3029J, Ipswich, Mass.) was transformed with the fusion protein DNA ligated into the Pet22b vector through heat shock and plated onto a lysogeny broth (LB) agar plate with 100 μg/ml ampicillin and grown overnight at 30 degrees Celsius (° C.). Following incubation, a single transformed colony was picked with a sterile pipette tip and inoculated into 5 mL of LB medium and grown overnight at 30° C. in a shaker incubator set at 200 rotations per minute (RPM) for 18 hours. Following overnight growth, glycerol stocks were generated through a 1:1 dilution of the transformed SHuffle® culture in 50% glycerol and stocks stored at −80° C. Fusion proteins were expressed by removal of a glycerol stock from −80° C. storage, and a small volume spread over an LB agar plate (100 μg/ml ampicillin) using a sterile inoculation loop. Following overnight incubation, a single colony was lifted from the LB agar plate and inoculated into a starter culture of LB medium with 100 μg/mL ampicillin in a shaker incubator (30° C., 200 RPM, 18 hours). Following an 18-hour incubation, the starter culture was diluted 1/100 into LB medium containing 100 μg/ml ampicillin and grown in a shaker incubator at 30° C., 200 RPM. Cell density was monitored at a wavelength of 600 nanometers (nm) using a spectrophotometer. Once the culture reached an optical density of 0.6-0.8, protein expression was initiated through the addition of 1 mM isopropyl β-d-1-thiogalactopyranoside (IPTG) into the growth medium with expression proceeding for 18-20 hours at 12° C., 200 RPM. Following expression, SHuffle® cells were pelleted through centrifugation at 10,000 relative centrifugal force (RCF) for 5 minutes. Pelleted cells were lysed using Bugbuster® (Millipore-Sigma, Burlington, Mass., 70584) protein extraction reagent containing 1 mg/ml lysozyme and 0.25 units/mL Benzonase® Nuclease (Millipore-Sigma, Burlington, Mass., 70584). The cell lysate, containing fusion protein, was passed over a 3 mL HisPur™ Ni-NTA spin column (Thermo Fisher Scientific, Waltham, Mass., 88226) through gravity filtration to allow purification through the C-terminal hexahistidine tag, encoded as part of the Pet22b vector. Non-specifically bound protein was removed from the column using manufacturer recommendations for wash buffer composition and volume. Following washing, the fusion protein was eluted using a 500 mM imidazole elution buffer. Following elution, the fusion protein was buffer exchanged into phosphate buffered saline buffer pH 7.4 (PBS) using a 5 mL, 7 kDa molecular weight cut-off Zeba™ spin desalting column (Thermo Fisher Scientific, Waltham, Mass., 89891). The final purified product in PBS was evaluated using sodium dodecyl sulfate—polyacrylamide gel electrophoresis (SDS-PAGE).

### Characterization of 2Rs15d-ColH Enzyme Activity

A fluorescence-based plate assay was developed to determine the enzyme activity of purified fusion protein. The collagenase substrate fluorescein pig skin gelatin (1 mg per vial) was purchased from Thermo Fisher Scientific (Waltham, Mass., D12054) and diluted into distilled water (dH2O) at 1 mg/ml. 20 microliters (μL) of the fluorescein substrate was added to individual wells of a 96 well Nunc MaxiSorp™ plate (Thermo Fischer Scientific, Waltham, Mass., 439454) and diluted with 80 μL of a buffer containing 50 mM tris(hydroxyethyl)aminomethane, 150 mM sodium chloride, 5 mM calcium chloride pH 7.6. A standard curve of collagenase activity was generated through serial dilution of a commercially obtained ColH (Worthington Biochemical, Lakewood, N.J., LS005273) with known activity between 0.05-5 units/milliliter (U/mL) in activity buffer. 100 μL of each ColH standard was run in duplicate with duplicate samples of 10×, 100× and 1000× dilutions of the fusion protein and immediately placed into a SpectraMax i3 multi-mode microplate reader (Molecular Devices, San Jose, Calif.). The fluorescence was read every 30 seconds for 5 minutes at an excitation wavelength of 485 nm and an emission wavelength of 530 nm. A standard curve of enzyme activity was generated using the observed change in fluorescence/minute for the ColH standards. The fusion protein dilution that fell within the linear range of the standard curve was used to determine the number of enzyme units of the purified product.

### Surface Plasmon Resonance

Surface plasmon resonance (SPR) was performed using a Reichert SR7500DC SPR and SR8100 autosampler (Reichert, Depew, N.Y.). HER2-Fc (Sino Biological, Beijing, China, 10004-H02H) or mouse serum albumin (MSA) (MyBioSource, San Diego, Calif., MBS135633) was immobilized onto a carboxymethyl dextran SPR chip (Reichert, Depew, N.Y., 13206066) using 1-Ethyl-3-(3-dimethylaminopropyl)carbodiimide/N-hydroxysuccinimide linker chemistry. A mobile phase of PBS 0.05% Tween-20 at a flow rate of 25 μL/min was used for all binding evaluations. Fusion protein dilutions were injected for 90 seconds, and the chip regenerated following dissociation with a 2-minute injection of a 10 millimolar (mM) glycine buffer pH 1.5. HER2 binding kinetics for 2Rs15d-ColH-ABD was evaluated at concentrations of 25, 50, 100, 200 and 500 nanomolar (nM) with a 60-minute dissociation time. HER2 binding kinetics for 2Rb17c-ColH-ABD was assessed at concentrations 10, 25, 50, 100, 250 nM with a 5-minute dissociation. MSA binding for 2Rs15d-ColH-ABD was evaluated at concentrations of 7.5, 15, 30 and 60 nM with a 10-minute dissociation time. MSA binding for 2Rb17c-ColH-ABD was assessed at concentrations of 7.5, 37.5 and 75 nM with a 10-minute dissociation time. The observed sensor-grams were fit to a 1:1 Langmuir binding model in the Scrubber analysis software to obtain the association rate constant (kon), dissociation rate constant (koff), and the equilibrium dissociation constant (K_{D}).

### Plasma Pharmacokinetics of 2Rs15d-ColH and 2Rs15d-ColH-ABD

Male Swiss-Webster mice were intravenously injected through the penile vein with 2Rs15d-ColH-ABD and 2Rs15d-ColH at a dose of 2000 collagenase units/kilogram bodyweight (U/kg) (3 mice/group). Blood samples were collected at 5, 15, 30, and 60 minutes after injection with lithium heparin as the anti-coagulant. Blood samples were centrifuged for 5 minutes at 500 RCF and plasma collected by pipetting. Plasma samples were analyzed for collagenase activity using the fluorescence collagen assay described above with the following changes. The activity of the 5-minute timepoint for 2Rs15d-ColH-ABD and 2Rs15d-ColH was determined using a 60-minute incubation with a standard curve of enzyme activity between 1-10 U/mL. The enzyme activity of the 15-minute time point for 2Rs15d-ColH-ABD and 2Rs15d-ColH and the 30-minute time point for 2Rs15d-ColH-ABD was determined using a 3-hour incubation with ColH standards between 0.25 and 2.5 U/mL. The 30-minute time point for the 2Rs15d-ColH and the 60-minute time point for 2Rs15d-ColH-ABD and 2Rs15d-ColH was analyzed following an 18-hour incubation with a standard curve between 0.025 and 0.5 U/mL. Area under the curve (AUC) values between 5 and 60 minutes were calculated for individual mice using the linear trapezoidal method.

### Fluorescent Assessment of Trastuzumab Distribution

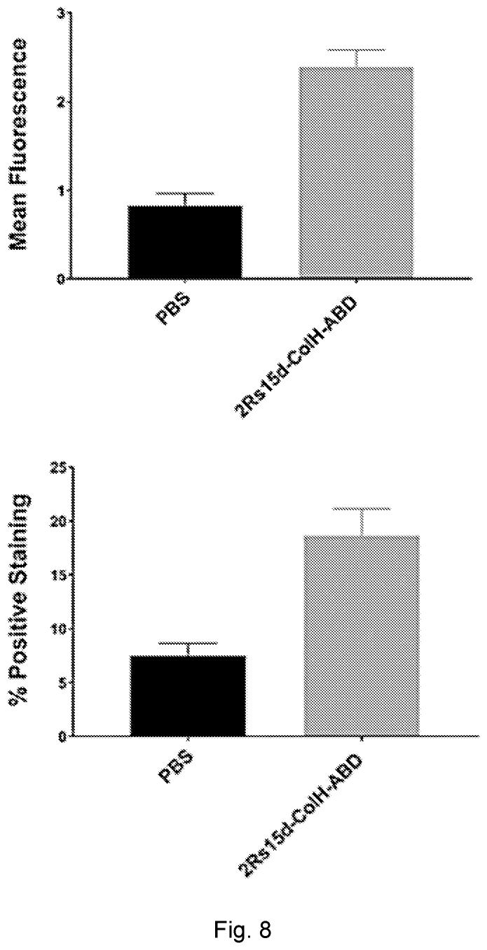

Trastuzumab was labeled with Alexa-Fluor 680 (Thermo Fisher Scientific, Waltham, Mass., A20188) following manufacturer recommendations with the recommended antibody concentration, and the volume doubled to limit over modification. NU/J mice bearing NCI-N87 xenograft tumors were administered 2 mg/kg of Alexa-Fluor680 labeled trastuzumab through retro-orbital injection. Ten minutes after trastuzumab administration, 2000 U/kg 2Rs15d-ColH-ABD035 was administered via penile vein injection with control mice administered a volume equivalent of PBS (2 mice/group). Mice were sacrificed 24 hours post-injection, tumors resected, covered in OCT freezing media (VWR, Radnor, Pa., 25608-930) and frozen in isopentane cooled liquid nitrogen. Frozen tumors were sectioned using an HM525 cryostat (Microm, Walldorf, Germany) at a slice thickness of 10 μM. Tumor sections were outlined with a PAP pen (Newcomersupply, Middleton, Wis., 6505) and covered with 1% mouse plasma in PBS buffer with a 1:50 dilution of a rat anti-mouse CD31 antibody (Invitrogen, Carlsbad, Calif., 390) labeled with Alexa-Flour555 (Thermo Fisher Scientific, Waltham, Mass., A20187). Tumors sections were stained for 30 minutes, followed by three five-minute PBS washes. Tumor sections were mounted in FluorSave (Millipore-Sigma, Burlington, Mass., 345789) and imaged identically using an EVOS Fl autofluorescent microscope (Thermo Fisher Scientific, Waltham, Mass.) with red fluorescent protein and cyanine 5 excitation cubes. Three slices were imaged per tumor and the mean fluorescence and percent area above threshold determined in ImageJ (NIH, Rockville, Md.). The mean of three slices was used to represent an individual tumor and statistical significance between the two groups determined in GraphPad Prism 7 (GraphPad, San Diego, Calif.) using Student's t-test. Selected tumor images that are shown in FIG. 8 were window/leveled identically in ImageJ for image clarity.

### Fluorescent Assessment of Tumor Collagen

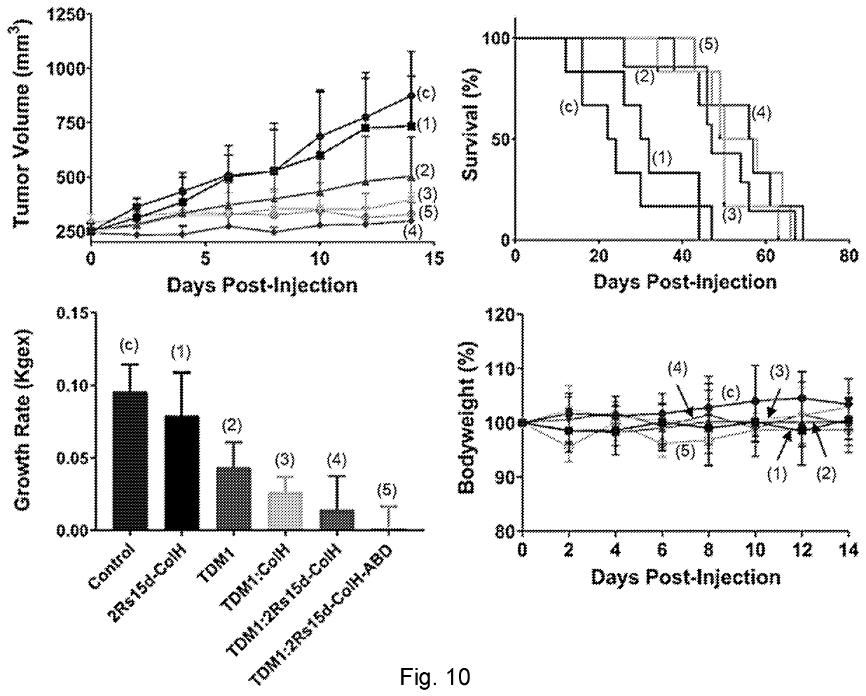

Male NU/J mice bearing NCI-N87 xenograft tumors were administered either PBS (n=1) or 2000 U/kg 2Rs15d-ColH-ABD035 (n=1) by penile vein injection. 8 hours after injection, mice were sacrificed, and tumors resected, frozen, and cryo-sectioned following the above protocol. Tumor collagen and vasculature were immunofluorescently stained for 1-hour using a 1:100 dilution of an Alexa-Flour680 conjugated anti-collagen antibody (Invitrogen, Carlsbad, Calif., PA5-29569) and a 1:50 dilution of a rat anti-mouse CD31 antibody (Invitrogen, Carlsbad, Calif., 390) labeled with Alexa-Flour555 (Thermo Fisher Scientific, Waltham, Mass., A20187). Tumors were washed, mounted and imaged following the above protocol. For image clarity, the images shown in FIG. 10 were window/leveled identically in ImageJ.

### NCI-N87 Efficacy

Mice bearing NCI-N87 xenografts at a volume of 250 mm³ were split into six treatment groups of (i) PBS vehicle (ii) 2000 U/kg 2Rs15d-ColH (iii) 1.8 mg/kg T-DM1 (iv) 1.8 mg/kg T-DM1 and 2000 U/kg ColH, (v) 1.8 mg/kg T-DM1 and 2000 U/kg 2Rs15d-ColH and (vi) 1.8 mg/kg T-DM1 and 2000 U/kg 2Rs15d-ColH-ABD. All groups had a total of six mice except for group iii, which had 7 mice. T-DM1 or PBS was administered via retro-orbital injection, and collagenase/PBS administered 10 minutes after the T-DM1/PBS administration via penile vein injection. Mice were observed for any signs of distress or bruising at the site of injection. Tumors were measured with digital Vernier calipers and volumes calculated using the formula L²×W/2, were L is the longest diameter of the tumor and W the shortest. Upon reaching a tumor volume of 1200 mm³ mice were sacrificed. Tumor volumes for individual mice, up to 14 days after injection, were fit to a mono-exponential growth function in GraphPad Prism 7. Best fit growth function (Kgex) values were compared between the groups with Student's t-test and Bonferroni's correction for multiple comparisons.

### Results

### Fusion Protein Expression and Functional Activity

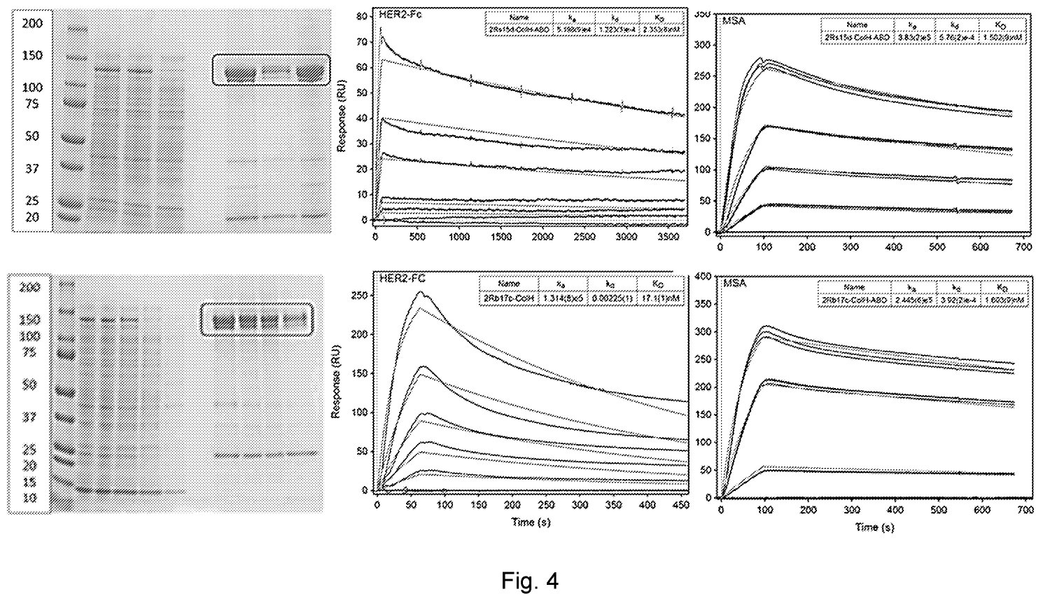

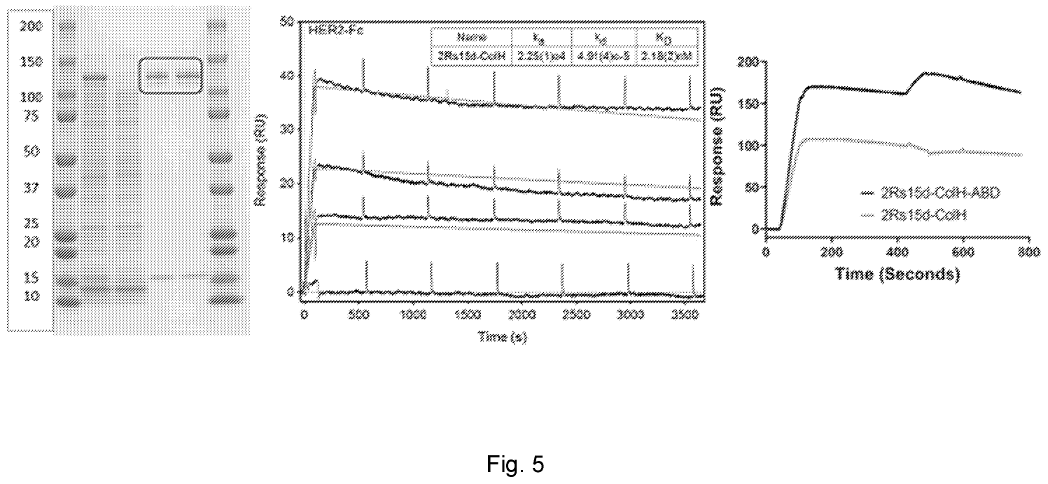

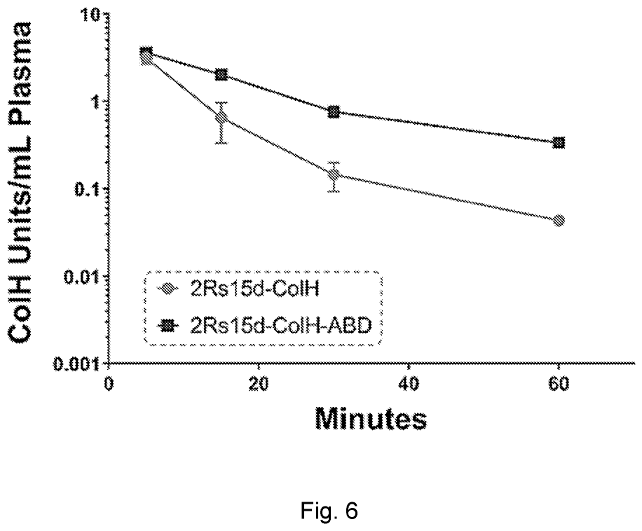

2Rs15d- and 2Rb17c-ColH-ABD were successfully expressed in E. Coli. SDS-PAGE gels for the expression and purification for both constructs are shown in FIG. 5. Contaminating bands that are the result of ColH auto-degradation are observed for both constructs. Degradation products causing erroneous experimental results were not a concern as gelatin zymography indicated only the intact protein is catalytically active. Collagenase activity was assessed using the described fluorescent assay with both constructs having enzyme activity of ˜500 units/mg. Surface plasmon resonance was used to assess HER2 and MSA binding. Identical MSA binding was observed, whereas 2Rs15d-ColH-ABD bound HER2 with a higher affinity (K_{D}=2.35±0.01 nM) than 2Rb17c-ColH-ABD (K_{D}=17.1±0.1 nM) (FIG. 4). As a result of the higher HER2 binding affinity and lack of competition of 2Rs15d with trastuzumab for HER2 binding, the 2Rs15d-ColH-ABD construct was chosen as the lead fusion protein. Collagenase is efficiently removed from the plasma by alpha-2-macroglobulin; therefore, it was unknown if albumin binding would significantly extend the half-life of 2Rs15d-ColH-ABD. The 2Rs15d-ColH-ABD construct was synthesized with a TEV protease site between the ColH and ABD domains (FIG. 6) with the intent of generating a 2Rs15d-ColH construct without the ABD. Significant auto-degradation of 2Rs15d-ColH-ABD is observed following prolonged incubation, limiting the utility of the TEV protease site. The ABD genetic sequence was removed by restriction enzyme digestion and 2Rs15d-ColH expressed as a separate fusion protein. 2Rs15d-ColH was produced in good yield and purity with similar HER2 binding affinity as 2Rs15d-ColH-ABD (FIG. 5). The successful removal of the albumin-binding domain is demonstrated in the far-right panel of FIG. 5. No increase in binding signal is observed when MSA is injected over 2Rs15d-ColH bound to HER2, in-contrast to 2Rs15d-ColH-ABD in which a signal increase is observed during the MSA injection at ˜400 seconds.

### 2Rs15d-ColH-ABD and 2Rs15d-ColH Plasma Pharmacokinetics

2Rs15d-ColH and 2Rs15d-ColH-ABD were administered to Swiss-Webster mice at a dose of 2000 U/kg and plasma activity determined at 5-, 15-, 30- and 60-minutes following administration. Plasma time profiles for both constructs are shown in FIG. 4. 2Rs15d-ColH-ABD and 2Rs15d-ColH demonstrated rapid plasma elimination; however, 2Rs15d-ColH-ABD retained significantly greater enzyme activity (p=0.001) with an AUC_{(5-60 minutes)} of 65.4±3.0 units×minute while 2Rs15d-ColH had an AUC_{(5-60 minutes)} of 27.7±6.9 units×minute. Based on the superior plasma pharmacokinetics 2Rs15d-ColH-ABD was chosen for additional analysis.

Impact of 2Rs15d-ColH-ABD on Trastuzumab Uptake and Tumor Collagen Content

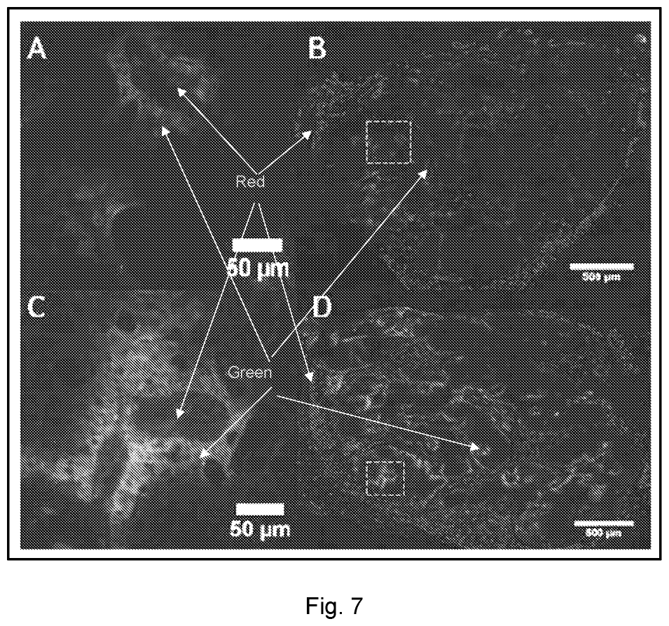

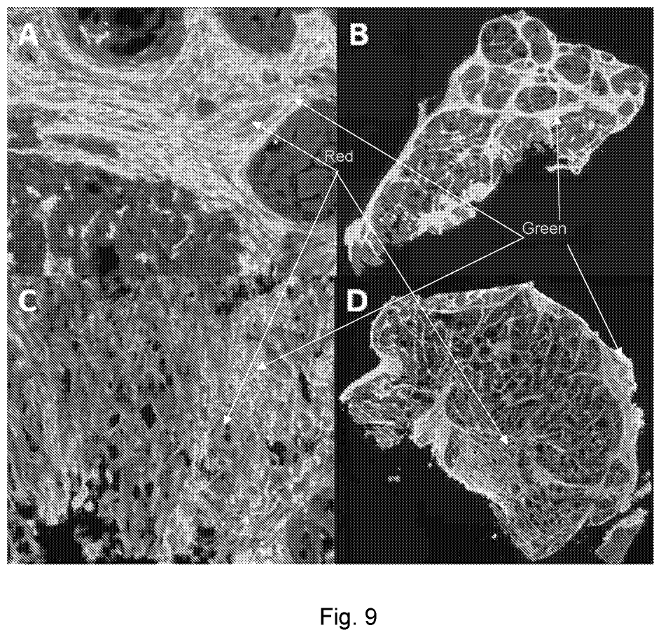

Co-administration of 2Rs15d-ColH-ABD dramatically increased the uptake of AF680-trastuzumab in NCI-N87 xenografts in comparison to AF680-trastuzumab administered alone (FIG. 7). 2Rs15d-ColH-ABD increased the trastuzumab fluorescence by 2.9-fold (p=0.01) and increased the percent of the total tumor area with trastuzumab positive staining by 2.5-fold (p=0.03) (FIG. 8). Interestingly, the tumor vasculature for 2Rs15d-ColH-ABD treated tumors stained brighter than the control tumors, whereas tumor vasculature surrounding the tumor slices stained identically (FIG. 7). This may be the result of CD31 epitopes on vasculature endothelium being occluded from antibody binding by peri-vasculature collagen. Administration of 2Rs15d-ColH-ABD may lead to removal/modification of the peri-vasculature collagen, increasing the accessibility of CD31 binding sites. To determine if a clear impact of 2Rs15d-ColH-ABD on tumor collagen could be observed, NCI-N87 tumors collected from mice administered only PBS or 2Rs15d-ColH-ABD were sectioned and tumor collagen labeled. An extensive collagen network can be observed in the control tumors (FIG. 9) with a majority of tumor vasculature appearing collapsed within the collagen network. Tumor collagen appears dispersed and less ordered in tumors treated with 2Rs15d-ColH-ABD, and several regions with larger vasculature radii are observed (FIG. 9).

### Impact of ColH on T-DM1 efficacy in NCI-N87 Xenografts

T-DM1 led to a significant increase in group survival in comparison to PBS or 2Rs15d-ColH alone (p<0.05). Likely resulting from the small group size and high intra-group variability, a significant extension in survival was not observed between the T-DM1 groups (FIG. 9). Observed tumor volumes for individual mice up to 14 days post-injection were fit to an exponential growth function, and the groups compared with Students t-test (FIG. 7). Co-administration of T-DM1 with untargeted ColH and 2Rs15d-ColH led to non-significant decreases in the mean growth rate in comparison to T-DM1 alone (p>0.01). 2Rs15d-ColH-ABD co-administration significantly (p=0.007) decreased the growth rate (kgex=0.002±0.014 day⁻¹) in comparison to T-DM1 alone (kgex=0.044±0.017 day⁻¹). Co-administration of the ColH constructs with T-DM1 did not significantly decrease the mean group bodyweight as shown in the bottom right panel of FIG. 10.

While the invention has been described through illustrative examples, routine modifications will be apparent to those skilled in the art, which modifications are intended to be within the scope of the invention.

1. A fusion protein comprising a collagenase and a single domain antibody (sdAb) which is specific for HER2.

2. The fusion protein of claim 1, wherein the sdAb has a sequence that is at least 85% identical to the sequence set forth in SEQ ID NO:3 and the collagenase has a sequence that is at least 85% identical to the sequence set forth in SEQ ID NO:5.

3. The fusion protein of claim 1, wherein the fusion protein comprises a sequence that is at least 85% identical to the sequence set forth in SEQ ID NO:1.

4. The fusion protein of claim 1, wherein the sdAb binds to an epitope of HER2 that is distinct from the epitope targeted by trastuzumab, epitope targeted by pertuzumab, or both.

5. The fusion protein of claim 1, further comprising an albumin binding domain (ABD).

6. The fusion protein of claim 5, wherein the fusion protein comprises a sequence that is at least 85% identical to the sequence set forth in SEQ ID NO:9 or SEQ ID NO:10.

7. The fusion protein of claim 1, wherein amino acid linkers are present between the amino acid sequences of sdAb and the collagenase, and collagenase and ABD.

8. A pharmaceutical composition comprising the fusion protein of claim 1.

9. A method of treating a tumor comprising administering to an individual in need of treatment a composition of claim 8.

10. The method of claim 9, wherein the individual is further administered an antibody directed to HER2, or an antibody-drug conjugate, wherein the antibody in the antibody-drug conjugate is directed to HER2.

11. The method of claim 10, wherein the composition is delivered at or near a tumor.

12. The method of claim 10, wherein the composition is delivered intratumorally.

13. The method of claim 9, wherein the composition is delivered intravenously.

14. The method of claim 10, wherein the antibody directed to HER2 is trastuzumab or pertuzumab.

15. The method of claim 10, wherein the antibody-drug conjugate is T-DM1.

16. The method of claim 10, wherein the fusion protein and the antibody or the antibody-drug conjugate are administered in the same composition.

17. The method of claim 10, wherein the fusion protein and the antibody or the antibody-drug conjugate are administered in separate compositions.

18. The method of claim 10, wherein the tumor is a solid tumor.

19. The method of claim 18, wherein the tumor is breast, pancreatic, gastric, intestinal, ovarian, colorectal, brain, lung, or liver.

20. The method of claim 18, wherein the tumor comprises a dense collagen-containing extracellular matrix.
